(19) United States
(12) Patent Application Publication
(10) Pub. No.: US 2023/0233257 A1
(43) Pub. Date: Jul. 27, 2023

(54) AUGMENTED REALITY HEADSET SYSTEMS AND METHODS FOR SURGICAL PLANNING AND GUIDANCE

(71) Applicant: PolarisAR, Inc., Miami, FL (US)

(72) Inventors: Aaron Young, Wylie, TX (US); Thien Nguyen, Fredrick, CO (US); Paul W. Mikus, Miami, FL (US)

(21) Appl. No.: 18/160,264

(22) Filed: Jan. 26, 2023

Related U.S. Application Data
(60) Provisional application No. 63/476,854, filed on Dec. 22, 2022.
(60) Provisional application No. 63/323,444, filed on Mar. 24, 2022.
(60) Provisional application No. 63/303,370, filed on Jan. 26, 2022.

(51) Int. Cl. A61B 34/10; A61B 90/00; A61B 34/20; A61B 34/00

(52) U.S. Cl. CPC A61B 34/10; A61B 90/36; A61B 34/20; A61B 34/25; A61B 2090/365; A61B 2034/107; A61B 2034/2055; A61B 2034/2068; A61B 2034/252

(57) ABSTRACT

Examples of systems and methods described herein may utilize augmented reality headsets in generating intra-operative plans and providing surgical guidance. An augmented headset positioned to view anatomical landmarks may receive position information for the anatomical landmarks based on a position of a pointer having fiducials, generate a surgical plan based on the position information. Using the augmented reality headset, information to guide a resection device based on the surgical plan may be displayed.

This application claims the benefit under 35 USC 119(e) of the earlier filing dates of U.S. Provisional Application 63/303,370, filed Jan. 26, 2022, U.S. Provisional Application 63/323,444, filed Mar. 24, 2022, and U.S. Provisional Application 63/476,854, filed Dec. 22, 2022. The aforementioned applications are all incorporated herein by reference in their entirety for any purpose.

### BACKGROUND

Augmented reality (AR) and navigation systems have found application in surgical settings. For example, some surgical navigation systems use pre-operative imaging of the anatomy of the patient subject to a surgical intervention, such as computer assisted tomography (CAT) imaging, magnetic resonance imaging (MRI), X-rays, etc. An AR surgical navigation system may be used to register or align the pre-operative imaging with live, intra-operative view of the anatomy. The pre-operative imaging and the live imaging may be displayed to a medical provider such as a surgeon. In some examples, the pre-operative imaging may be overlaid on live intra-operative images of the anatomy of the patient to help the medical provider plan and/or execute a surgical intervention.

Surgical navigation systems are utilized to provide surgeons with assistance in identifying precise locations for surgical applications of devices, targeted therapies, instrument or implant placement or complex procedural approaches. The benefit of surgical navigation is that it allows for information that can be utilized to improve almost any surgical intervention. A challenge in current navigation systems is the reliance on pre-operative images to reconstruct the anatomical landscape as the basis for surgical planning. The pre-operative images are static representations of the anatomy and may in some cases not align with the anatomy at the time of surgery. Moreover, as the surgical intervention occurs, changes in the anatomical landscape may occur and are not taken into account by a static pre-operative plan. Another challenge in current navigation systems is the potential for interference with the line of sight between the cameras used to capture the images of the markers disrupting the referencing and ultimately the navigation of the process as a whole. Current systems have a large footprint within the operating room (OR) and require the surgeon to look away from the surgical field. Head-mounted systems, on the other hand, have no footprint other than the head-mounted display (HMD) and use optical cameras that are essentially aligned with the surgeon's point of view.

Augmented reality and mixed reality (AR/MR) have gained increased interest in the medical field in recent years. The use of AR/MR typically employs an HMD that superimposes content to whatever the user sees. The location of where to superimpose an image can be determined by use of markers or trackers placed in the environment. This common approach to using AR/MR allows reference information in the superimposed content to be geolocated at a point in actual environment that may allow the user to access during the time of surgery. For example, if a pretreatment Computed Tomography (CT) scan of a patient is converted to a three-dimensional (3D) image and used to plan the procedure, both the 3D reconstructed image and plan can be superimposed over the patient's actual knee. This would allow the surgeon to compare the pretreatment plan to place an implant visually against the actual knee. The approach assumes the ability to register the superimposed image to the actual knee with enough precision to make the visual overlay clinically useful. If the superimposed image is overlaid on the actual anatomy with precision, it can be manipulated to allow for adjusting its position. So the pretreatment plan can be moved in a variety of directions in a manner that allows for visualizing the impact of changing the location of the initial plan.

The current approach is thus primarily limited to superimposing plans created using imaging methods like CT, Magnetic Resonance, Fluoroscopy or X-ray. The imaging modalities result in a visual or digital model of the patient's pre-operative targeted anatomy that can be used for measurement of the environment for purposes of precise location of an object. The superimposed information can be an accurate representation of areas of a patient's body. For example, a CT image is likely to reveal precise measurements of hard tissues like bone structures. Thus superimposition of a knee image reconstructed and displayed on an actual knee should match closely if not exactly, provided the knee is exposed and all other surrounding tissues have been removed. In practice, this represents a limitation for superimposing plans created by imaging methods. The need to expose the exact layers they are displaying rarely occurs. So, the position of the overlay needs to be inferred to the actual location of the anatomy to be potentially useful.

### BRIEF DESCRIPTION OF THE DRAWINGS

FIG. 1 is a schematic illustration of a system arranged in accordance with examples described herein.

FIG. 2 is a schematic illustration of a head-mounted display arranged in accordance with examples described herein.

FIG. 3 illustrates an example of a medical provider marking a feature of a patient's anatomy using an example of the surgical planning system of FIG. 1.

FIG. 4 is a schematic illustration of aspects of an intra-operative plan which may be generated by example systems arranged in accordance with examples described herein.

FIG. 5 is an illustration of a medical provider placing a resection guide in accordance with surgical guidance provided in accordance with examples of systems and methods described herein.

FIG. 6 is a flow chart of a method of determining a pose of an instrument of the system of FIG. 1.

FIG. 7 is a schematic view of a method using a headset to display content on anatomy arranged in accordance with examples described herein.

FIG. 8 is a flowchart of a method of performing a surgical intervention with the system of FIG. 1.

FIG. 9A is an image of a display which may be presented to a medical provider through an augmented reality headset in accordance with examples described herein.

FIG. 9B is an image of a view from an augmented reality headset during an example of a landmarking process arranged in accordance with examples described herein.

FIG. 10A shows images of views from an augmented reality headset showing landmarks of a femur in accordance with examples described herein.

FIG. 10B is an image of a view from an augmented reality headset during an example of a landmarking process of a femur in accordance with examples described herein.

FIG. 10C is an image of a view from an augmented reality headset during an example of a landmarking process of a femur in accordance with examples described herein.

FIG. 10D is an image of a view from an augmented reality headset during an example of a landmarking process of a femur in accordance with examples described herein.

FIG. 10E is an image of a view from an augmented reality headset during an example of a landmarking process of a femur in accordance with examples described herein.

FIG. 10F is an image of a view from an augmented reality headset during an example of a landmarking process of a femur in accordance with examples described herein.

FIG. 10G is an image of a view from an augmented reality headset during an example of a landmarking process of a femur in accordance with examples described herein.

FIG. 10H is an image of a view from an augmented reality headset during an example of a landmarking process of a femur in accordance with examples described herein.

FIG. 10I is an image of a view from an augmented reality headset during an example of a landmarking process of a femur in accordance with examples described herein.

FIG. 10J is an image of a view from an augmented reality headset during an example of a landmarking process of a femur in accordance with examples described herein.

FIG. 10K shows images of views from an augmented reality headset showing landmarks of a tibia in accordance with examples described herein.

FIG. 10L is an image of a view from an augmented reality headset during an example of a landmarking process of a tibia in accordance with examples described herein.

FIG. 10M is an image of a view from an augmented reality headset during an example of a landmarking process of a tibia in accordance with examples described herein.

FIG. 10N is an image of a view from an augmented reality headset during an example of a landmarking process of a tibia in accordance with examples described herein.

FIG. 10O is an image of a view from an augmented reality headset during an example of a landmarking process of a tibia in accordance with examples described herein.

FIG. 10P is an image of a view from an augmented reality headset during an example of a landmarking process of a tibia in accordance with examples described herein.

FIG. 10Q is an image of a view from an augmented reality headset during an example of a landmarking process of a tibia in accordance with examples described herein.

FIG. 10R is an image of a view from an augmented reality headset during an example of a landmarking process of a tibia in accordance with examples described herein.

FIG. 10S is an image of a view from an augmented reality headset during an example of a landmarking process of a tibia in accordance with examples described herein.

FIG. 10T is an image of a view from an augmented reality headset during an example of a landmarking process of a tibia in accordance with examples described herein.

FIG. 11 is an image of a view from an augmented reality headset of an evaluation interface depicting parameters relevant for a knee implant in extension and flexion.

FIG. 12 is an image of a view from an augmented reality headset of a balancing interface allowing for adjustment of a knee implant in accordance with examples described herein.

FIG. 13A shows several views of a resection marker for tracking a resection guide in accordance with examples described herein.

FIG. 13B shows a view of a resection marker for tracking a resection guide in accordance with examples described herein.

FIG. 13C shows a view of a resection marker for tracking a resection guide in accordance with examples described herein.

FIG. 13D shows several views of the resection marker attached to resection guides in accordance with examples described herein.

FIG. 14A is a schematic illustration of a femur and tibia having markers and a resection guide reversibly coupled to a marker arranged in accordance with examples described herein.

FIG. 14B is a schematic illustration of a femur and tibia having markers and a resection guide reversibly coupled to a marker arranged in accordance with examples described herein.

FIG. 14C is a schematic illustration of a femur and tibia having markers and a resection guide reversibly coupled to a marker arranged in accordance with examples described herein.

FIG. 15A is a flow chart of a method of detecting fiducials of a marker of the system of FIG. 1.

FIG. 15B is a flow chart of a method of estimating sub-pixel centers of fiducial edges of FIG. 15A.

FIG. 16A is an image of a view from an augmented reality headset of a resection interface allowing for adjustment of a resection guide to match a planned resection plane.

FIG. 16B is an image of a view from an augmented reality headset of a resection interface allowing for adjustment of a resection guide to match a planned resection plane on a femur in accordance with examples described herein.

FIG. 17 is an image of a view from an augmented reality headset of a resection interface allowing for adjustment of a resection guide to match a planned resection plane on a tibia in accordance with examples described herein.

FIG. 18 is an image of a view from an augmented reality headset of a resection interface allowing for adjustment of a resection guide to match a planned resection plane in 4-in-1 resection in accordance with examples described herein.

### DETAILED DESCRIPTION

Certain details are set forth herein to provide an understanding of described embodiments of technology. However, other examples may be practiced without various of these particular details. In some instances, well-known circuits, AR/VR technology, surgical operations, control signals, timing protocols, and/or software operations have not been shown in detail in order to avoid unnecessarily obscuring the described embodiments. Other embodiments may be utilized, and other changes may be made, without departing from the spirit or scope of the subject matter presented here.

Existing systems for use of AR in a surgical setting that involve registering a pre-operative image to live anatomy have drawbacks that limit their use. For example, pre-operative imaging or a plan based thereon becomes obsolete the moment a surgeon makes the first cut or incision, thereby changing the anatomy of the patient such that the anatomy no longer matches the pre-operative imaging. For example, in a total knee replacement, a surgeon may resect portions of the condyles and/or the patellar surface of the femur and/or the condyles of the tibia. Once the surgeon cuts the bone, the anatomy of the femur and tibia no longer match the pre-operative imaging and any plan based on the pre-operative imaging is no longer relevant to additional resections made to the anatomy. In addition, pre-operative plans need to maintain constant registration to the anatomy to accurately reflect the position of the plan to the live anatomy. Movement of the live anatomy that is not captured and corrected for can provide false positioning of the plan to the targeted anatomy. Improved methods and systems for intra-operative planning that can account for changes in the anatomy during the progression of a surgical intervention are needed.

The challenge of starting with a reconstructed image as the basis for overlay of a plan on anatomy is not just the overlay but the idea of the static nature of the plan itself in contrast to the dynamic environment that surgery is. Surgeons have used pretreatment or intra-operative imaging as a point in time snapshot that is constantly changing during surgery. So reliance on a static image for planning is inherently limited by the extent of the changes made during surgery. If a surgical plan calls for resection or reconstruction of an anatomical site, once the surgeon makes the first cut, the imaging used to plan that cut and others going forward has become potentially obviated due to the change in the anatomy as a result of the intervention.

There is a need for an ability to dynamically adjust a plan and place surgical instrumentation that adapts to the surgical environment throughout more (or the whole) of a surgical procedure. Moreover, there is a need to develop systems and methods for intra-operative planning that do not rely, or place less reliance, on pre-operative imaging.

Examples of systems and methods are described herein that utilize a headset for measurement and planning of or for a surgical environment. Examples of systems and methods described herein may further utilize a pointer in conjunction with the headset. The headset may receive position information for anatomical landmarks based on a position of the pointer. The surgical plan may be generated based on the position information. Information to guide a surgical device based on the surgical plan may be displayed using the headset.

The measurement and planning of a surgical environment may use a defined coordinate system for the purposes of locating and navigating optimal placement of surgical instruments without the need to reference or superimpose imaging methods like CT, X-ray, MRI and Fluoroscopy. Examples may utilize a headset or other system that can image the surgical environment with one or more of a variety of camera types. The headset can also employ a number of sensors including depth sensors for measurements that may be useful in understanding the position of the anatomy in the environment. The resulting camera images and measurements can be utilized to form a surgical plan and/or a reconstruction of the actual anatomy without the aid of additional imaging modalities. Markers affixed to the anatomy or elsewhere in the environment can be used in conjunction with the headset to create a coordinate system. For example, fiducials may be attached to each marker, and the coordinate system may be created based in part on the fiducials. The fiducials, may for example, be arranged in such a manner that they define a plane, and therefore a coordinate system. A map of the anatomy and/or a surgical plan may accordingly be generated and displayed in the coordinate system defined by the markers, and aligned with landmarks identified by the pointer.

To obtain position information for landmarks, the headset may track a position of a pointer relative to the marker. The movement of the pointer may be used to identify a landmark. For example, the headset may recognize that the pointer had slowed, stopped, contacted, or otherwise indicated a particular landmark. The headset may accordingly obtain the position of the landmark based on the position of the pointer when the landmark is indicated. An intra-operative plan can be generated based on the position of the landmark. Surgical guidance using the headset may be provided based on the intra-operative plan.

An advantage of examples of this approach over methods and systems utilizing superimposed pretreatment plans in some examples may be the ability to plan based on updated anatomy during the procedure compared to the pretreatment static environment displayed. Note also the ability for a surgeon or other practitioner to indicate landmarks using the headset. The headset may advantageously allow the individual performing the landmarking to move their viewpoint to access the landmark location in a desired manner. By positioning both a fixed marker and the pointer indicating the landmark in a field of view of a camera of the headset, the location information of the landmark in the coordinate system of the marker may be obtained by the headset. The surgeon or other practitioner is able to freely move the camera about (e.g., by moving their body, and therefore the headset to obtain this view). Accordingly, in some examples, the use of the headset and markers to identify relevant anatomy may improve accuracy because the anatomy identification (e.g., landmarking) may be performed from the surgeon's point of view through the headset, allowing the surgeon to move around, reduce occluding objects, and more accurately identify the anatomy. Note also that multiple landmarks may be obtained, and may be obtained from different views by the surgeon. The surgeon may be in one position when identifying a first landmark, and another position when identifying another landmark, for example. In some examples, the use of the headset and markers to identify anatomy relevant to the surgical plan may reduce or eliminate a need to rely on pre-operative imaging, which may improve accuracy as well as reducing the burden in preparing for surgery, as pre-operative imaging may be less significant.

Examples of systems and methods are described herein that utilize a marker that includes an insertion member in conjunction with a resection guide. The resection guide may have a slot that may receive either the insertion member or a resection device. Accordingly, a resection guide may include a marker with one or more fiducials. Headsets described herein may accordingly determine a position of the resection guide in an environment and may provide guidance for placement of the resection guide. The resection guide may be placed in accordance with an intra-operative plan, the marker removed, and a resection made using a resection device moving through the slot.

Examples of systems and methods are described herein that utilize a pointer in conjunction with fiducials associated with a marker affixed to at least one of a femur or a tibia to detect positions of anatomical features in proximity to a knee. A planned resection plane for a body part proximate to a knee may be generated based on the location(s) of the anatomical features. An actual resection plane may be determined based on a view of a resection guide having a marker inserted in the guide from the headset. Thus, surgical guidance using the headset may be provided to position the resection guide in a manner to align the actual resection plane with the planned resection plane.

Examples of methods and systems described herein may have particular benefits over existing systems in that a model of the patient's anatomy and/or a surgical plan may be updated in the surgical theater. For example, once a resection is made, the plane of the completed resection can be measured relative to the planned resection plane or line. For example, even if a resection is made with some deviation from the plan, the remainder of the planned resections can be adjusted accordingly based on the position of the completed resection plan or line. In existing systems, an inaccurate cut could propagate throughout the anatomy as one resection after another is based on an uncorrected model of the patient's anatomy.

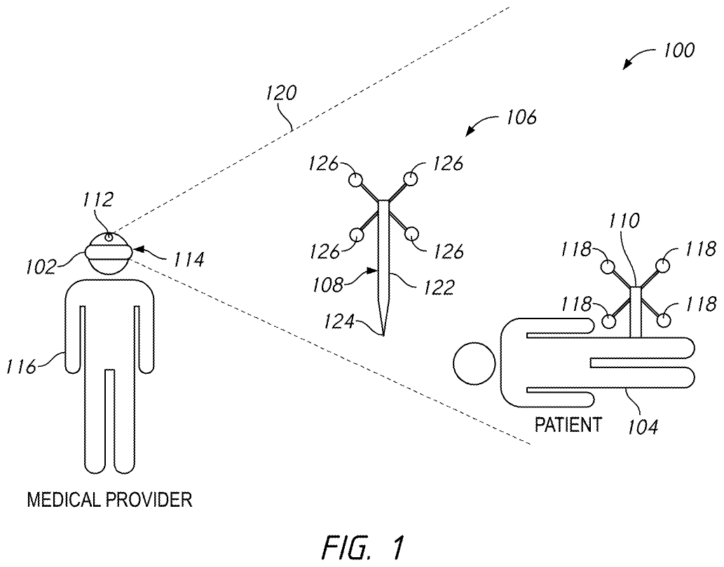

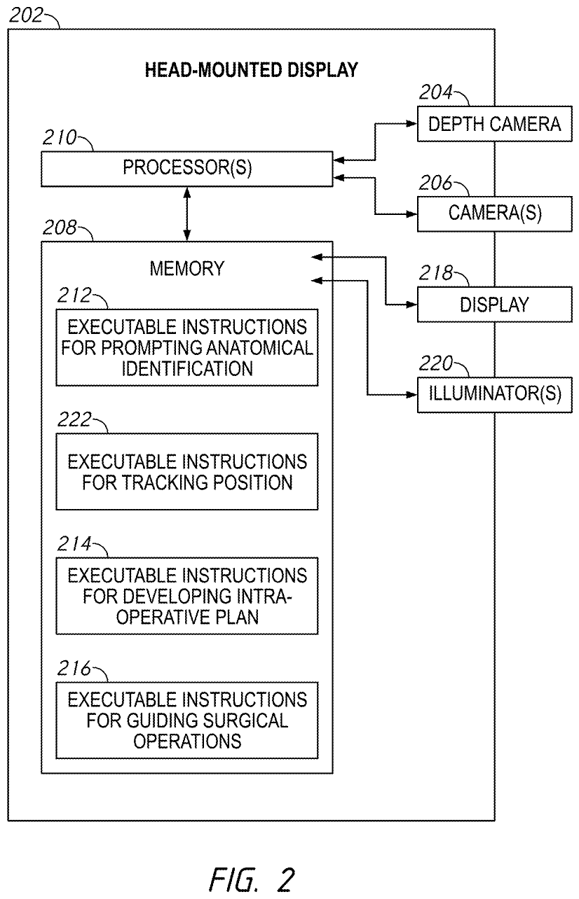

FIG. 1 is a schematic illustration of a system arranged in accordance with examples described herein. The system 100 includes a headset 102, which may be worn by medical provider 116. The headset 102 may include a vision system 112. The vision system 112 may include one or more cameras such as a depth camera 204 in FIG. 2, and one or more head tracking cameras, eye tracking cameras, greyscale cameras, or other visible light or infrared cameras. Any (one or more) of the cameras may have a field of view 120 positioned to image a virtual environment 114. The system 100 further includes a pointer 108. The pointer 108 may include a plurality of fiducials 126. The pointer 108 may include a stylus 122 coupled to the fiducials 126 and may have a tip 124 for pointing. The system 100 further includes a marker 110. The marker 110 may include a plurality of fiducials 118. The marker 110 may be affixed to an anatomy of patient 104. The system 100 of FIG. 1 is exemplary. Additional, fewer, and/or different components may be used in other examples.

Examples of systems described herein accordingly include a headset, such as the headset 102. The headset 102 may be implemented using an augmented reality headset, such as, but not limited to, a MICROSOFT HOLOLENS device, a META PLATFORM OCULUS device, and/or a MAGIC LEAP device. The headset 102 may be worn by a medical provider, such as the medical provider 116. The medical provider 116 may be a surgeon; however, in some examples the medical provider 116 may be a surgical assistant, physician's assistant, anesthesiologist, registered nurse, or other being involved in the surgical field. The headset 102 may image a field of view 120 and present a virtual environment 114 to the medical provider 116. The headset 102 may accordingly include one or more cameras, as discussed with respect to the vision system 112.

Examples of systems described herein may include one or more markers, such as the marker 110. The markers may be placed in a fixed position on or proximate a patient, such as the anatomy of the patient 104. The patient 104 may be a human, a dummy and/or model (e.g., in the case of training systems), an animal, and/or any other surgical subject. The marker 110 may be positioned at a location particular to a procedure to be performed, in some examples. While a single marker 110 is shown in FIG. 1, any number may be used including 2, 3, 4, 5, 6, 7, 8, 9, or more markers. The markers may be trackable and/or locatable by the headset 102, such as sensors through optical targets, radio frequency, or other mechanisms.

The markers may have one or more fiducials, such as the fiducials 118. The fiducials 118 may be positioned and designed to allow for the headset 102 to determine a coordinate system of the marker 110 and/or determine a position of the marker 110 in the coordinate system. For example, the fiducials 118 may be optical targets that may reflect a particular wavelength or band of wavelengths of light. The fiducials 118 may be arranged in known locations (e.g., a predetermined pattern) about the marker 110. For example, four fiducials 118 may be positioned at respective quadrants around an end of the marker 110. Additionally, each fiducial 118 may be reflective and/or otherwise patterned or shaded for identification by the headset 102. In the example shown, the fiducials 118 are spherical and/or circular. In other examples, the fiducials 118 may be any suitable shape which allow for detection and localization of the marker 110. In some examples, in this manner, by identifying the position of the fiducials 118, the headset 102 may determine the position of the marker 110 attached to the headset 102, in a sensor (e.g., camera) coordinate system, such as a simultaneous localization and mapping (SLAM) coordinate system, relative to the headset 102 from a single image. In some examples, a coordinate system may be defined by a plane created or indicated by the fiducials. The marker 110 may include one or more fixtures such as a screw, clamp, adhesive, or other fastener adapted to attach the marker 110 to the patient 104 (e.g., to a bone or other components of the patient 104). The distances of the fiducials 118 with respect to a body of the marker 110 may be known.

Generally, systems described herein may utilize at least one or more markers. The markers may be used to establish the camera coordinate system defined by the marker positions. For example, the headset 102 may be used to identify the positions (e.g., locations and orientations) of the markers, including the marker 110 of FIG. 1 and another marker attached to the patient 104 (not shown in FIG. 1). Fiducials attached to the markers, such as the fiducials 118 of FIG. 1, may be used to determine the location and orientation of the marker 110. During operation, a position of the pointer 108 may be determined, e.g., by the headset 102, in the camera coordinate system defined by the markers. In some examples, one marker may be placed on a femur of a patient and another marker placed on a tibia of a patient. In this manner, a coordinate system of a femoral marker may be obtained, and a coordinate system of a tibial marker may be obtained. The headset may further understand a translation between the femoral and tibial coordinate systems (e.g., by capturing a view of both markers).

In some examples, the headset 102 may simultaneously build a map of surroundings of the medical provider 116 in an environmental coordinate system. The headset 102 may track itself in six degrees of freedom within the map. The map may be used for rendering holograms near the medical provider 116 or other objects of interest. When two markers are present, the positions and orientations of the markers of each are used in order to compute transformation between the camera and environmental coordinate systems associated with each other for a time of capturing the single frame including the markers. In addition, the headset 102 may provide transformation from the camera coordinate system to the environmental coordinate system for each frame while tracking of the headset 102 is active.

In some examples, a medical provider may make one or more incisions in the skin, facia, or other soft tissues of a patient to reveal bony structures of the patient's anatomy. The medical provider may affix one or more markers, such as a marker 110, to the bony structures. For example, the medical provider may affix one marker to a tibia of the patient and another to the femur of the patient. For example, a tibial marker affixed to a tibia may be used to identify and/or locate a location of one or more tibial landmarks in the map. For example, a femoral marker affixed to a femur may be used to identify and/or locate one or more femoral landmarks in the map. Estimating a location of a certain portion of a body part, such as a femoral head center (hip center), in conjunction with the marker affixed to the body part, such as the femur, may use the map built by the headset 102. By tracking the position and/or orientation of a marking instrument (e.g., a pointer) in the real environment, the medical provider can have the headset generate a model of the anatomy and may update that model intra-operatively as the anatomy changes during the surgery (e.g., due to resection of bones). The marking instrument may be tracked in the environmental coordinate system established by the markers fixed to the bony structure. For example, the medical provider may have the marking instrument point to the body part of the patient's anatomy and define features thereof. In the non-limiting example of a knee replacement, a medical provider may have a marking instrument, such as a pointer, to indicate landmarks, such as features of the femur, tibia, fibula, hip, ankle, etc. In some examples, responsive to slowed or stopped motion of the marking instrument for a predetermined period, the headset may detect identification of the landmarks, and may determine positions of the landmarks in the coordinate system. The headset may automatically generate a model of the anatomy, based on determining a position of the landmark in the coordinate system, and generate an intra-operative plan based on the positions of the landmarks. As the medical provider resects bone, the anatomy of the patient changes and the surgeon can easily update the model intra-operatively by touching a newly formed feature of the bone. The surgeon then can proceed with the surgical intervention, updating the model as needed.

Note that, in examples described herein, the medical provider may mark and/or indicate features which may or may not be in an initial field of view of the headset. For example, a medical provider may be prompted to indicate a feature which is occluded from the provider's headset's current field of view. The provider may move their head such that the headset's field of view changes to include the indicated feature, and use a marking instrument (e.g., a pointer) to indicate the feature. In this manner, a greater number of features may be available for collection by the medical provider than if a fixed camera system had been used, and occluded areas unavailable. Moreover, the medical provider may move their body and/or head such that the headset is within a particular distance and/or range of the desired feature to be indicated (e.g., within 6 inches to 2 feet in some examples). This may be closer than is achievable with a fixed camera system having a view of an entire patient or operating room. Accordingly, by placing the headset in an appropriate position for each feature to be indicated, accuracy of a position determination for the feature may generally be improved. For example, a medical provider may position themselves such that a first feature is centered or positioned in some other location of their field of view, and then indicate the first feature using a marking instrument. The medical provider may move their body and/or head to another position such that a next feature is centered or positioned in some other location of their field of view, and then indicate the second feature using a marking instrument. By changing the field of view for particular feature indications, accuracy may be improved and/or it may be possible to mark otherwise occluded features.

Examples of systems described herein may include one or more pointers, such as the pointer 108. The pointer 108 may be a device which may include a stylus 122 having a tip 124. One or more fiducials 126 may be positioned in known locations (e.g., a predetermined pattern) about the stylus 122. In this manner, the pointer 108 may be trackable by the headset 102. The fiducials 126 may be analogous to the fiducials 118 and may be optical targets trackable by the headset 102. The pointer 108 may include a stylus, pointer, or other similar implement that can be easily grasped by the medical provider 116 and that includes a tip 124, that may function as a pointer marker, suitable to precisely indicate one or more anatomical features. The fiducials 126 may be affixed to the stylus 122 with one or more arms such that the fiducials 126 are spaced from one another in a known pattern. A distance of each of the fiducials 126 from the tip 124 of the stylus 122 may be known. In the example of FIG. 1, the fiducials 126 are positioned in four quadrants about an end of the pointer 108.

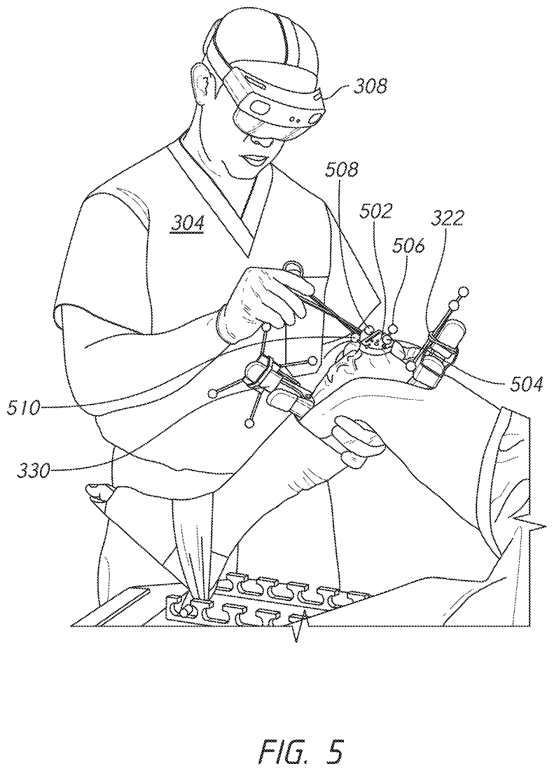

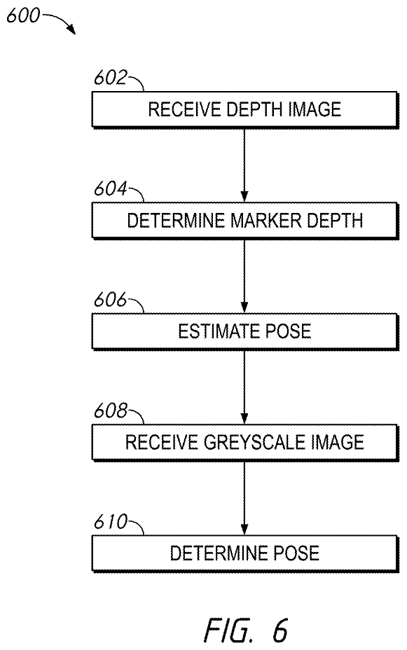

The systems disclosed herein may track a position of a fiducial using one or more cameras of the vision system 112. For example, the headset 102 may use a depth camera to first estimate a pose of the marker 110 or the pointer 108 and then use a greyscale or other image from the depth camera or another camera to more accurately resolve the pose of the marker or pointer 108. See, for example, the description relating to FIG. 6 herein.

FIG. 2 is a schematic illustration of a head-mounted display 202 arranged in accordance with examples described herein. The head-mounted display 202 may include one or more processor(s) 210 coupled to memory 208. The one or more processor(s) 210 may include, for example, a central processing unit (CPU), a reduced instruction set computing (RISC) processor, a complex instruction set computing (CISC) processor, a graphics processing unit (GPU), a digital signal processor (DSP) such as a baseband processor, an application specific integrated circuit (ASIC), another processor, or any suitable combination thereof. The memory 208 may include main memory, disk storage, or any suitable combination thereof. The memory 208 may include, but are not limited to, any type of volatile or non-volatile memory, a non-transitory computer readable medium such as dynamic random-access memory (DRAM), static random-access memory (SRAM), erasable programmable read-only memory (EPROM), electrically erasable programmable read-only memory (EEPROM), Flash memory, solid-state storage, etc. The memory 208 may include executable instructions for prompting anatomical identification 212, executable instructions for tracking position 222, executable instructions for developing intra-operative plan 214, and/or executable instructions for guiding surgical operations 216. The head-mounted display 202 may further include depth camera 204, camera(s) 206, display 218, illuminator(s) 220, and/or a speaker (not shown) coupled to the processor(s) 210 and/or memory 208. The head-mounted display 202 may be used to implement and/or be implemented by the headset 102 of FIG. 1. The components shown in FIG. 2 are exemplary only. Additional, fewer, and/or different components may be used in other examples. Generally, the head-mounted display system of FIG. 2, and/or other computing system(s), may perform any computations described herein. For example, the computer readable media shown in FIG. 2 may include executable instructions for performing computations described herein.

The head-mounted display 202 may be an augmented reality device, such as a MICROSOFT HOLOLENS device, a META PLATFORM OCULUS device, and/or a MAGIC LEAP device. Accordingly, the head-mounted display 202 may allow a wearer to view a field of view in a scene in an environment around the wearer, and the head-mounted display 202 may display various information and/or objects in the scene. While described as a head-mounted display, in some examples, other augmented reality devices may additionally or instead be used, such as a mobile or cellular telephone (e.g., a smartphone), a tablet, a watch, or other electronic devices. While described as a head-mounted display, in some examples the head-mounted display 202 may be worn or carried by another part of a body.

Head-mounted displays described herein may include one or more depth cameras, such as depth camera 204. The depth camera 204 may be implemented using a time-of-flight camera or sensor which may provide depth information to portions of a scene based on a measurement of a time for a round trip of a light pulse to reflect off an object in the scene and return to the depth camera 204. In some examples, the depth camera (e.g., a time-of-flight camera) 204 may be implemented using light detection and ranging (LIDAR) or continuous wave technology. The depth camera 204 may have a viewing angle that changes with the view of the wearer of the head-mounted display 202. In some examples, the wearer of the head-mounted display 202 (e.g., the medical provider 116 of FIG. 1) may gaze in a direction such that the field of view of the depth camera 204 encompasses a view of the fiducials 118 of the pointer 108 and/or the marker 110 of FIG. 1. Accordingly, head-mounted displays may obtain depth information to various objects in a scene. For example, the depth camera 204 may obtain a distance to one or more fiducials described in the system of FIG. 1, such as fiducials 118 and/or fiducials 126. Additionally or instead, the depth camera 204 may obtain distances to various portions of a patient anatomy—e.g., a leg, foot, knee, arm, elbow, shoulder, or head.

Head-mounted displays described herein may include one or more illuminators, such as illuminator(s) 220. The illuminator(s) 220 may illuminate generally any wavelength of light, or combinations of wavelengths. In some examples, one or more illuminator(s) 220 may emit infrared light. The light emitted by one or more of the illuminator(s) 220 may be used by the depth camera 204 to obtain depth information (e.g., to measure a time for light emitted by one or more illuminator(s) 220 to reflect off an object in the scene). In some examples, one or more illuminator(s) 220 may be implemented using incoherent light sources such as light emitting diodes (LEDs). In some examples, one or more illuminator(s) 220 may be implemented using coherent light sources such as laser emitters and/or laser diodes.

Head-mounted displays described herein may include one or more additional cameras, such as camera(s) 206. The camera(s) 206 may include, for example, one or more color cameras, video cameras, greyscale cameras, or the like. In some examples, head-mounted displays described herein may include one or more additional sensors including, but not limited to, one or more accelerometers, gyroscopes, positional sensors, temperature sensors, wavelength sensors, or combinations thereof.

Head-mounted displays described herein may include one or more displays, such as the display 218. The display 218 may project an image of information and/or objects onto the scene viewed by the wearer of the head-mounted display 202. In this manner, selections may be presented to the wearer of the head-mounted display 202 and/or surgical plan information and/or guidance may be viewed by the wearer of the head-mounted display 202.

Head-mounted displays described herein may receive input from a wearer of the head-mounted display, such as the head-mounted display 202. Input may be received, for example, by sensing a direction of gaze of the wearer. In some examples, input may be received by detecting a position of a portion of the wearer (e.g., a finger may be used to select a displayed selection and/or a pointer, such as the pointer 108 of FIG. 1 may be used to provide input and/or select).

In examples described herein, the head-mounted displays may be used during and in preparation for and/or follow-up to surgical operations. The head-mounted displays may be used to select surgical operations, to gather tracking information, to formulate operative plans, and to provide guidance based on those plans. The operative plans may be monitored and/or adjusted during the surgical operation based on anatomical changes identified during the surgical operation. In this manner, it may not be necessary to utilize pre-operative images and/or to register pre-operative images to the anatomy viewed by the medical provider during the surgical operation. Rather, the information about the anatomy used to generate the operative plan, provide surgical guidance, and/or modify the operative plan may be gathered from views of the anatomy taken by the head-mounted display of the actual anatomy. Note also that the field of view can be selected and adjusted by the medical provider wearing the headset to obtain accurate anatomical information.

Accordingly, head-mounted displays described herein may be provided with hardware, firmware, and/or software for identifying anatomy, tracking markers, fiducials, and/or pointers or other objects, developing and/or modifying intra-operative plans, and/or providing surgical guidance.

In the example, of FIG. 2, the head-mounted display 202 may include the processor(s) 210 coupled to the memory 208. The memory 208 may be encoded with executable instructions (e.g., software) which, when executed by the processor(s) 210, allow the head-mounted display 202 to perform actions described herein, such as executable instructions for prompting anatomical identification 212, executable instructions for tracking position 222, executable instructions for developing intra-operative plan 214, and/or executable instructions for guiding surgical operations 216. The processor(s) 210 may be implemented using, for example, one or more controllers, microcontrollers (e.g., MCUs), custom circuitry (e.g., one or more field programmable gate arrays (FPGAs) and/or application specific integrated circuits (ASICs)), one or more central processing units (CPUs), graphics processing units (GPUs), or combinations thereof. The memory 208 may be implemented using random-access memory (RAM), read-only memory (ROM), or combinations thereof. It is to be understood that the processor and memory components of the head-mounted display 202 may be quite flexible. Moreover, the head-mounted display 202 may in some examples (e.g., through a wired and/or wireless communication interface not shown in FIG. 2) communicate with one or more other computing systems, such as one or more servers, computers, displays, monitors, or combinations thereof.

Examples of head-mounted displays described herein may be utilized to request anatomical identifications. For example, the head-mounted display 202 of FIG. 2 may include the executable instructions for prompting anatomical identification 212. During operation, a medical professional may identify a surgical procedure to be performed. The identification may be made, for example, by making a selection from multiple surgical procedures displayed to the medical professional by the head-mounted display 202. In some examples, the identification may be made previously and the head-mounted display 202 may have software dedicated to the identified procedure. Any of a variety of procedures may be supported by the systems described herein such as, but not limited to, knee replacement (e.g., total knee replacement), hip replacement, and/or shoulder replacement. The executable instructions for prompting anatomical identification 212 may prompt a medical professional to identify certain anatomy relevant to the identified procedure to be performed. For example, the executable instructions for prompting anatomical identification 212 may cause the head-mounted display 202 to display an indication of specified anatomy for the medical professional to identify. The anatomy may be relevant to the identified surgical procedure, and the location of the specified anatomy may be useful in generating an intra-operative plan.

The medical provider may then identify the anatomy as prompted. For example, the medical provider 116 of FIG. 1 may identify anatomy as requested by the headset 102. The anatomy may be identified by the medical provider 116 using the pointer 108 to indicate a location of the requested anatomy. For example, the medical provider 116 may place the pointer 108 so the tip 124 is on, adjacent, and/or proximate the requested anatomy. Any number of anatomical sites may be requested for identification, including 1, 2, 3, 4, 5, 6, 7, 8, 9, 10 or another number of anatomical sites.

Examples of anatomical features which may be requested by the head-mounted display 202 and/or identified by a medical provider include, but are not limited to, hip center (e.g., collect hip center via hip rotation), knee center (e.g., collect femoral knee center, such as the center of the femoral canal), ankle center (e.g., collect medial and lateral malleoli), and/or tibia landmarks (e.g., collect five tibial proximal landmarks, which may be in addition to malleoli, and visualize tibial axis independent of femur, such as not connected at femoral knee center). Other examples include identification of a sagittal plane (e.g., medical provider to outline and/or paint Whiteside's line to estimate sagittal plane) and/or identification of condyle extremes (e.g., medical provider to paint and/or outline distal condyle surfaces).

Examples of head-mounted displays described herein may locate and/or track objects, such as objects having fiducials. Accordingly, the head-mounted display 202 may include the executable instructions for tracking position 222. The executable instructions for tracking position 222 may cause the head-mounted display 202 to locate and/or track a position of one or more pointers and/or markers. For example, the location of pointer 108 may be tracked by the head-mounted display 202. The head-mounted display 202 may track the location of the pointer 108 relative to, for example, the marker 110. Accordingly, when the medical provider indicates a location of requested anatomy with the pointer 108, the head-mounted display 202 may identify the position of that anatomy, such as by identifying a position of that anatomy relative to the marker 110 and/or any other markers. In this manner, the head-mounted display 202 may identify a location of the requested anatomy in a camera coordinate system, e.g., a SLAM coordinate system, relative to the vision system. In some examples, the marker 110 itself may be positioned at a location of particular anatomy relevant for a surgical operation. Accordingly, the position of the marker 110 may itself be used to develop intra-operative plans. Position may be tracked using the depth camera 204, light emitted from the illuminator(s) 220, and the fiducials 118 and/or fiducials 126 on the pointer 108 and/or marker 110. The position and orientation of the pointer 108 and the marker 110 may be tracked using computer vision methods such as “projective-N-point” (PnP) techniques. The head-mounted display 202 may create a relative coordinate system between the pointer 108 and the marker 110 and/or other markers used in the system 100. Thus, the head-mounted display 202 can accurately determine the locations of features of the anatomy of the patient.

Accordingly, based on a selected and/or identified or otherwise known procedure, the head-mounted display 202 may prompt the medical provider 116 to touch one or more anatomical features with the tip 124 of the pointer 108. As the medical provider 116 touches the features, the head-mounted display 202 may register the feature in a model of the patient's anatomy. When the medical provider 116 has placed the tip 124 on, adjacent to, and/or proximate the prompted anatomical feature, the medical provider 116 may indicate to the head-mounted display 202 that the tip 124 is on the feature. The indication may be provided, for example, using a particular gesture, by holding the tip 124 in a single place for a particular time, by gazing in a particular direction, and/or by pressing a button, such as a button or other indicator on the stylus 122, or other tactile input. Other input mechanisms may be used, including auditory inputs, such as speaking a word to indicate the anatomy has been identified. The head-mounted display 202 may then record the feature in a relative coordinate plane between the pointer 108 and the one or more markers 110. In some examples, the feature may be a point on the anatomy, such as the lateral or medial condyle of the femur, etc. In some examples, the feature may be a line (e.g., Whiteside's line, a line that which runs from the center of the intercondylar notch to the deepest point of the trochlear groove anteriorly). In some examples, the feature may be a surface or area of the anatomy. In the examples where the feature is a line, area, or surface, the medical provider 116 may move the tip 124 along the feature (e.g., paint the feature) and/or outline the feature, and the depth camera 204 may record the locations of the tip 124 to generate a model of the position and orientation of the feature.

In some examples, the head-mounted display 202 may provide marker visibility verification. For example, the head-mounted display 202 may present a visualization of the tracked marker, such as the marker 110 while a medical provider is pointing to requested anatomy. A visualization of the tracked pointer, such as the pointer 108, may also be provided. The medical provider may be prompted by the head-mounted display 202 to verify the one or more markers, such as the marker 110, and remain visible when the pointer 108 is being used to identify anatomy.

Examples of head-mounted displays described herein may develop intra-operative plans. Accordingly, the head-mounted display 202 may include the executable instructions for developing intra-operative plan 214. The executable instructions for developing intra-operative plan 214 may cause the head-mounted display 202 to make certain calculations for a particular surgical procedure based on the identified locations of requested anatomy. For example, one or more planes, lines, volumes, or other relevant structures may be calculated based on the location of the requested anatomy. The intra-operative plan may include one or more positions for surgical instrument(s), cut lines, locations for positioning instruments or guides, or other planning actions.

Examples of calculations that the head-mounted display 202 may make in accordance with the executable instructions for developing intra-operative plan 214 include calculation of femoral implant (e.g., to adjust translation/rotation of femoral component, while visualizing resection planes), femur/tibia implant metrics (e.g., metrics for each individual implant, such as angles/distances relative to landmarks), gap metrics (e.g., visualize flexion/extension gap or implant articulation surface gap), distal femur resection (e.g., visual guidance of resection guide to planned distal resection), flexion angle (e.g., should start at 0 degrees in full extension, such as a measure of how much the knee is flexed), and metrics beyond just distal resection depth, e.g., rotation angles, etc., during femoral implant manipulation. Other calculations and plans may be used in other examples.

In some examples, following a medical provider's identification of tibial proximal landmarks, which may be in addition to malleoli, the head-mounted display 202 may calculate and display through the display 218 a tibial axis which may be independent of the femur, and may not be connected at the femoral knee center.

In some examples, metrics for implants, such as femur and/or tibia implants, may be calculated and/or displayed, such as various angles and/or distances to place the implant relative to one or more identified anatomical features (e.g., landmarks).

In some examples, gap metrics may be calculated and/or visualized through the display 218. For example, a flexion and/or extension gap may be calculated and/or visualized, and/or an implant articulation surface gap.

Examples of head-mounted displays described herein may provide surgical guidance based on intra-operative plans. For example, the head-mounted display 202 may include the executable instructions for guiding surgical operations 216. The executable instructions for guiding surgical operations 216 may cause the head-mounted display 202 to display guidance for a medical professional conducting a surgical operation. For example, a location of a cut line, and/or location to place a guidance or surgical instrument may be displayed by the display 218 overlaid on the surgical scene. As a surgeon places surgical instruments, guidance devices or other tools, the display 218 may display any of a variety of information which may aid in the accurate placement of such tools and devices. For example, the guidance devices and/or other tools may also be tracked (e.g., may be coupled to one or more fiducials which allow their position to be determined by the head-mounted display 202). A distance between the current position of the tool and a desired position of the tool may be displayed, and guidance as to in which direction or how to move the tool may be displayed by the display 218. Additionally, the executable instructions for guiding surgical operations 216 may further cause the head-mounted display 202 to provide the guidance for the medical professional conducting the surgical operation using the speaker (not shown).

In some examples, 4-in-1 resection guidance may be provided, such as by using the display 218 planar rotation and/or translation error within a distal resection plane.

In some examples, tibial resection guidance may be provided, such as by using the display 218 resection plane angular and depth error, analogous in some examples to distal femoral resection.

Examples of head-mounted displays described herein may modify intra-operative plans based on actions occurring during a surgical procedure. For example, after a particular step in a surgical operation (e.g., a cut), the executable instructions for prompting anatomical identification 212 may prompt a medical provider to identify additional anatomical features (e.g., an end of the cut, a plane exposed by the cut, etc.). The new anatomical feature position information may be used by the executable instructions for developing intra-operative plan 214 to modify and/or develop an additional portion of the pre-operative plan based on the newly located anatomical feature.

In some examples, the head-mounted display 202 may support a femoral workflow. In an example femoral workflow, the femoral workflow may optionally be selected from among a plurality of supported workflows—e.g., by the medical provider 116 using the head-mounted display 202. The executable instructions for prompting anatomical identification 212 may prompt the medical provider 116 to use the pointer 108 to identify features such as medial/lateral epicondyles, Whiteside's line, anterior cortex (e.g., for notch checking), posterior or distal medial/lateral condyle surfaces, etc. The executable instructions for developing intra-operative plan 214 may calculate signed medial/lateral distal condyle distances, signed medial/lateral posterior condyle distances, signed anterior cortex distance, varus/valgus alignment, flexion alignment, axial rotation, and/or axial plane translation. These metrics may be visualized and/or used to guide surgical operations regarding the femur.

In some examples, head-mounted display 202 may support a tibia workflow. In an example tibia workflow, the tibia workflow may optionally be selected from among a plurality of supported workflows—e.g., by the medical provider 116 using the head-mounted display 202. The executable instructions for prompting anatomical identification 212 may prompt the medical provider 116 to use the pointer 108 to identify the medial/lateral plateau base (e.g., through outlining and/or painting), the canal center, anterior tubercle, and/or PCL attachment center. Based on the identified anatomy, the executable instructions for developing intra-operative plan 214 may calculate varus/valgus alignment, posterior slope, medial/lateral plateau depth, axial rotation, and/or axial plane translation.

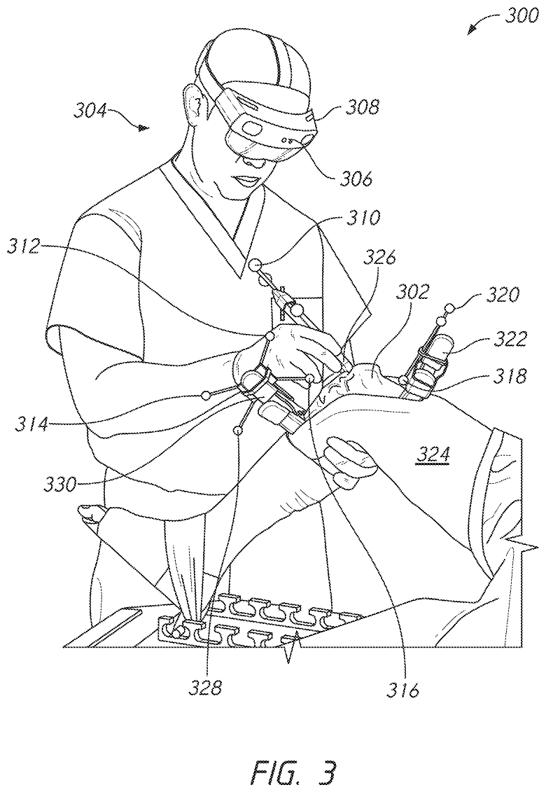

FIG. 3 illustrates an example of a medical provider marking a feature of a patient's anatomy using an example of the surgical planning system of FIG. 1. In the example of FIG. 3, a medical provider 304 is preparing to perform a surgical operation on a patient 324. The medical provider 304 is wearing headset 308, which may include depth camera 306. The medical provider 304 has placed marker 322 and marker 330 on the patient's femur and tibia, respectively. The marker 322 has fiducial 318 and fiducial 320. The marker 330 has fiducial 316, fiducial 312, fiducial 314, and fiducial 328. The medical provider 304 is holding pointer 326. The pointer 326 is connected to fiducial 310 and additional fiducials. The system in FIG. 3, such as the headset 308, marker 322, and pointer 326 may be implemented by and/or used to implement the system 100 of FIG. 1. For example, the headset 308 may be implemented by and/or used to implement headset 102 of FIG. 1 and/or head-mounted display 202 of FIG. 2.

In the example of FIG. 3, the medical provider 304 has selected to perform a knee replacement. In the example shown, the lower part of the leg (tibia, fibula and foot) is immobilized with respect to the upper part of the leg (femur 302). The soft tissue covering the patella has been opened and pulled back to reveal the bony features of the knee. The medical provider 304 may have been prompted by the headset 308 to place the marker 322 and marker 330 on the patient's leg bones. Next, the medical provider 304 may be prompted by the headset 308 to indicate Whiteside's line, which may be used by the headset 308 to estimate a sagittal plane of the knee.

In this manner, the headset 308 may determine a location of Whiteside's line and/or an estimate of the sagittal plane in a coordinate system tracked by the headset 308. The location may be relative to locations of the marker 322 and/or marker 330. Generally, systems described herein may utilize at least two markers in order to establish a relative coordinate system defined by the markers. Positions and/or orientations of pointers, fiducials, or other objects of the markers may be determined by the headset or other systems described herein in the relative coordinate system defined by the markers. The positions and orientations of the markers of each are used in order to compute a transformation between the relative coordinate system and an environmental coordinate system associated with each other at a time associated with a frame captured.

The following is a non-limiting example of anatomical features or clinical landmarks that may be identified, along with example reasons for the identification, in a total knee replacement procedure. In other procedures on the knee or other parts of the body, other landmarks relevant to the surgical intervention may be identified. An advantage of the disclosed methods and systems is to capture landmarks easily and sufficiently accurately for their use in providing surgical guidance. Based on the location of the landmarks, surgical guidance may be provided and intra-operative plans developed and/or modified during a surgical procedure. There may not be a need, accordingly, to register or utilize pre-operative imaging for surgical guidance or localization of any features.

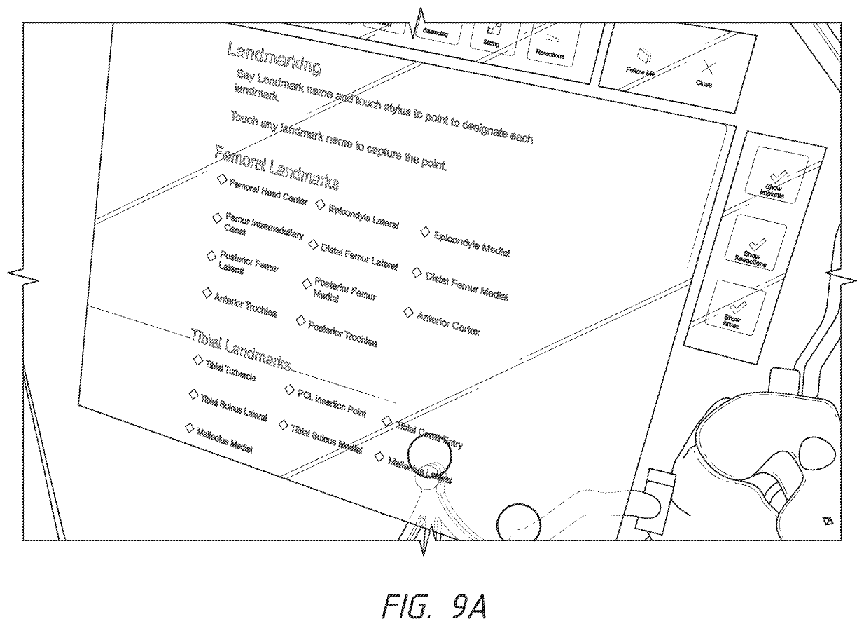

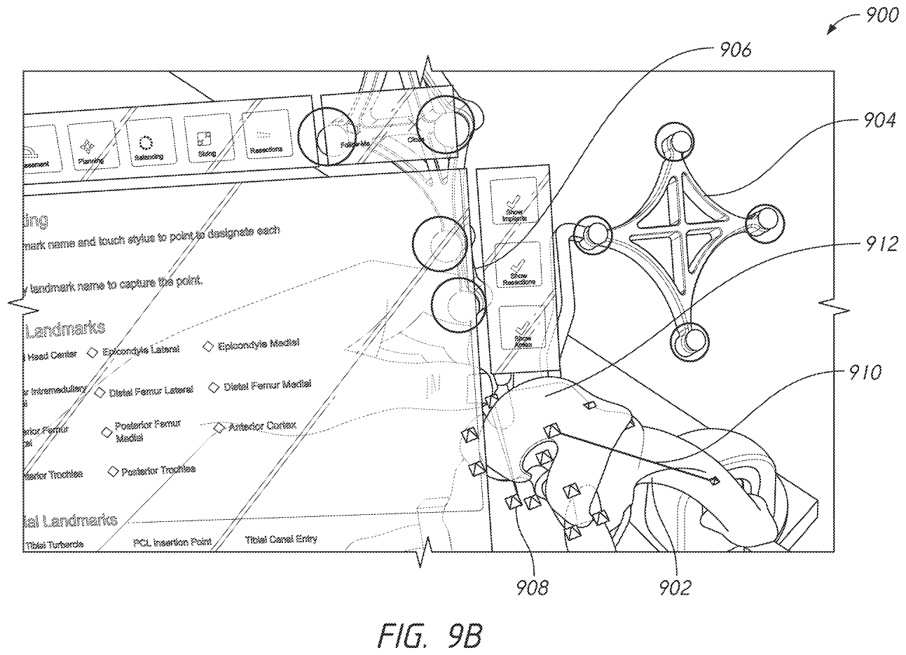

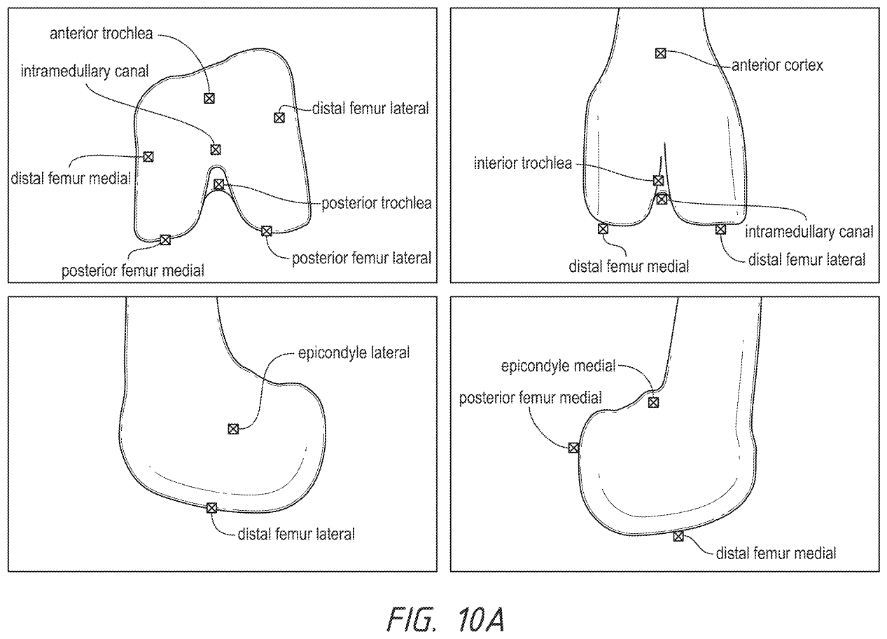

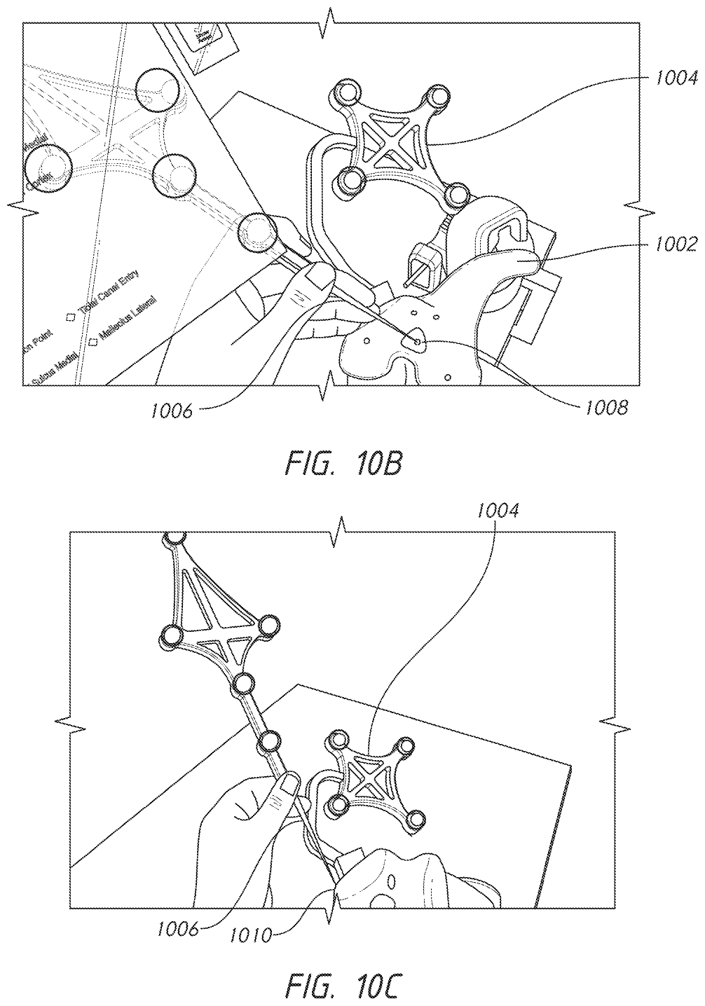

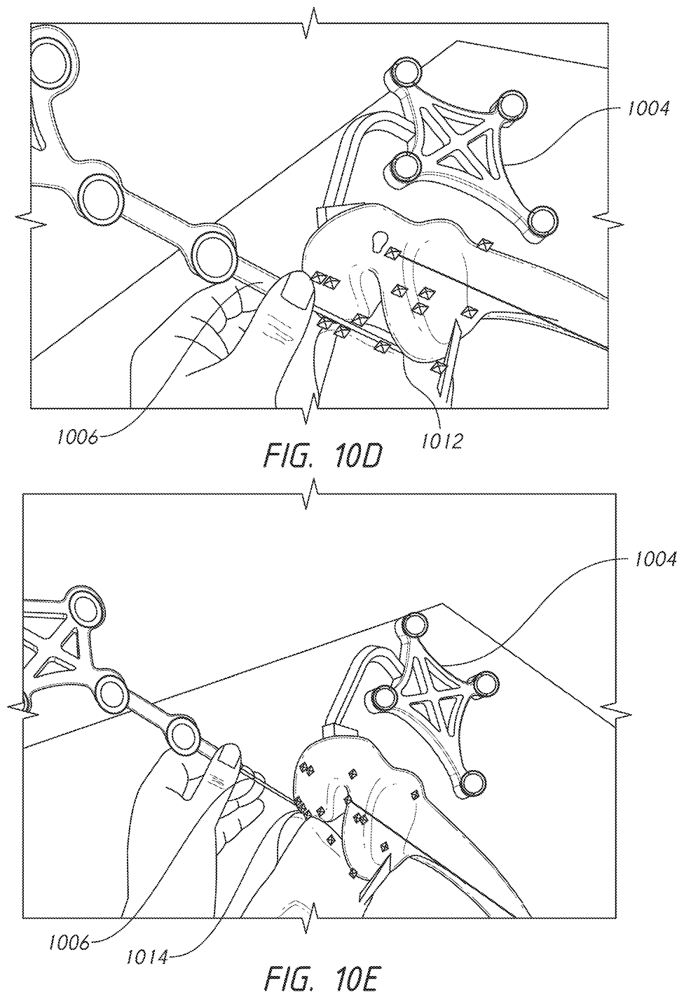

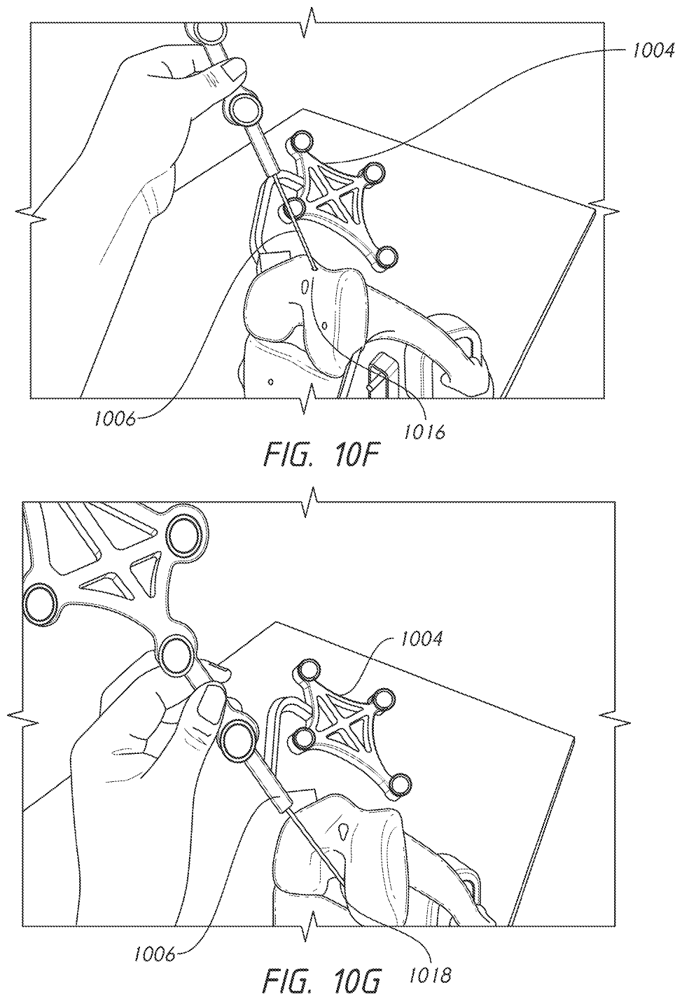

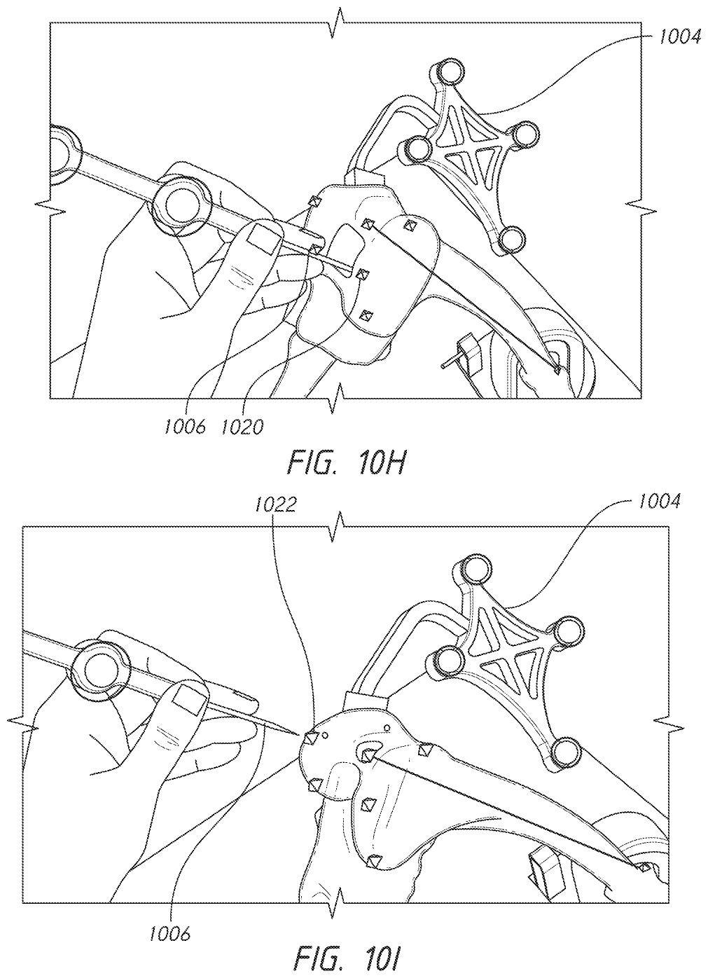

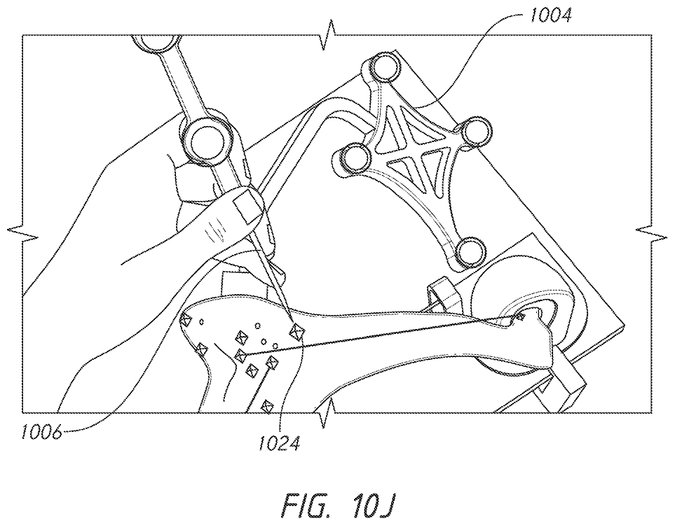

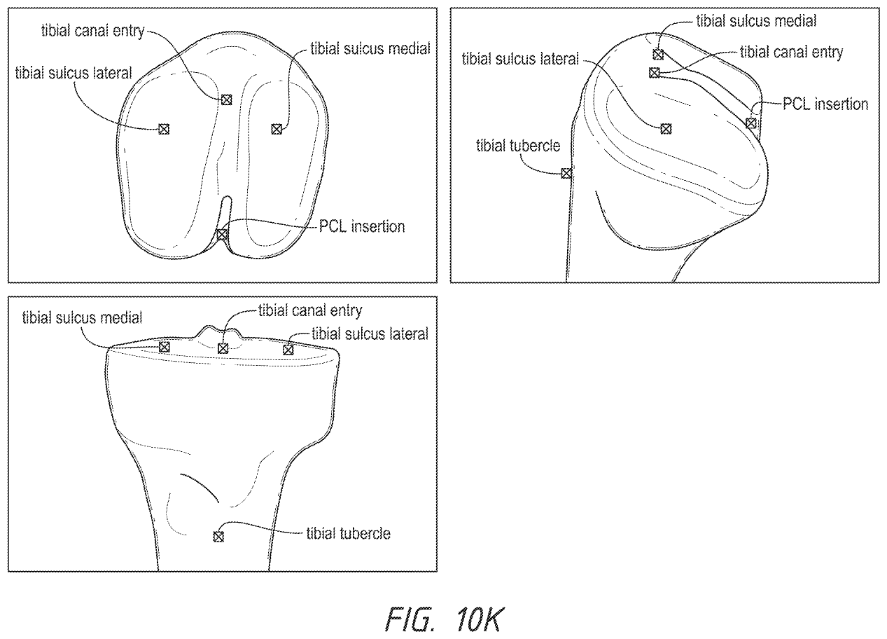

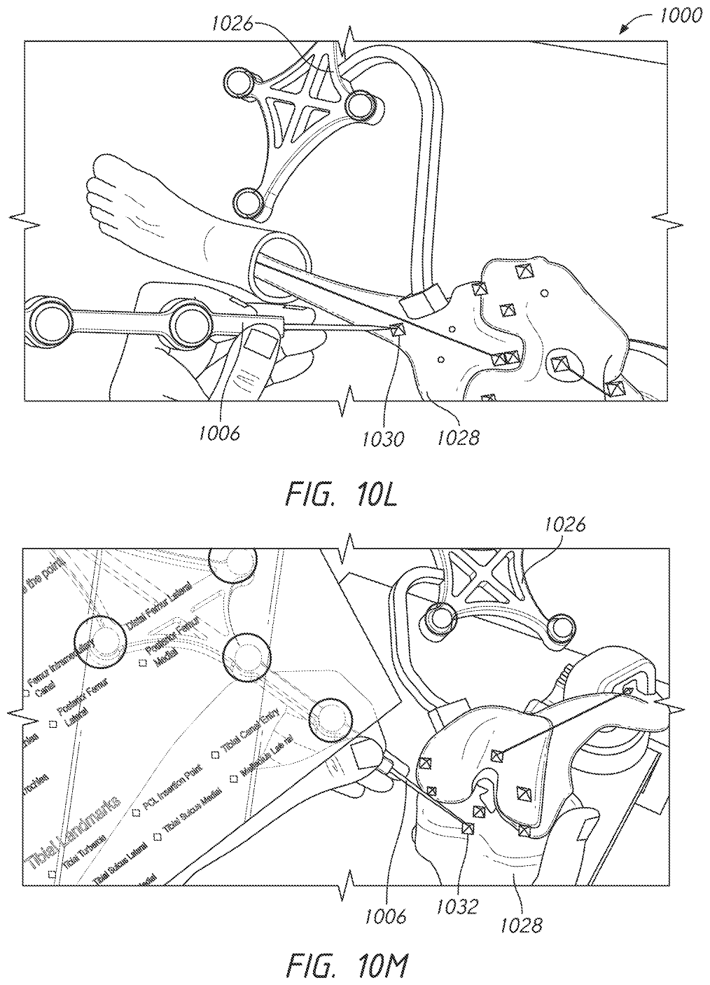

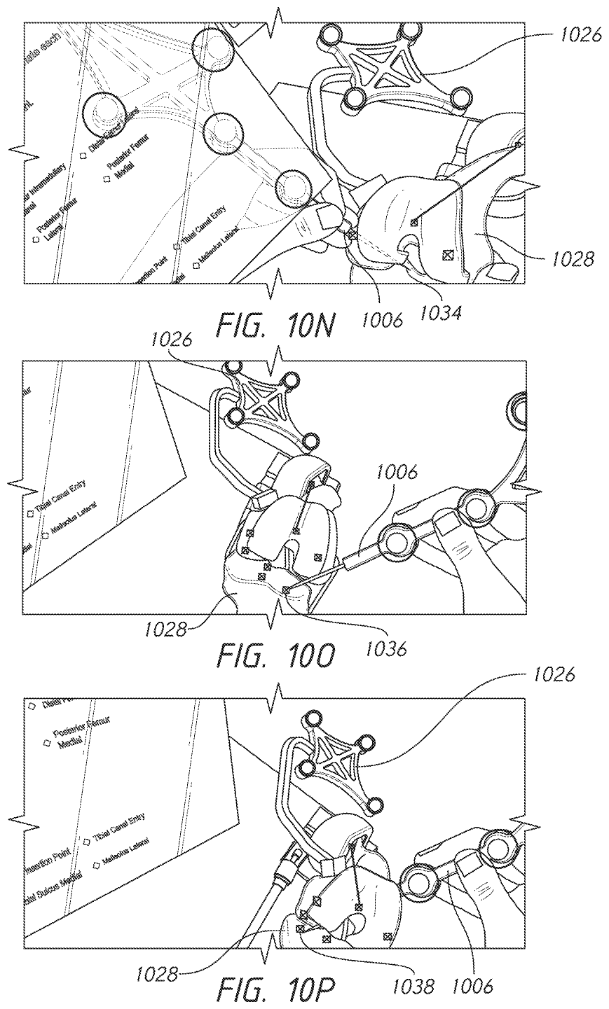

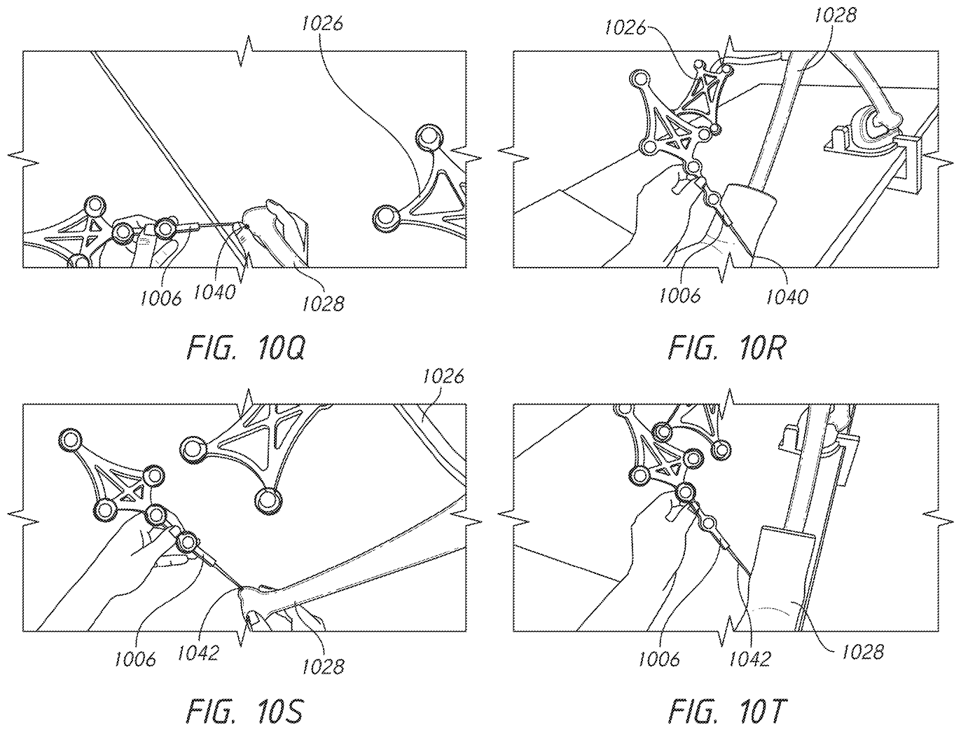

In the example of a total knee replacement, a medical provider may be prompted to provide femoral landmarking. Femoral landmarking may be used to gather information regarding the location and/or orientation of features that may be used in guiding the total knee replacement procedure. During a femoral landmarking procedure, a medical provider may be prompted (e.g., using a display of the headset 102 of FIG. 1) to mark and/or otherwise indicate a variety of features related to the femur. (See also FIGS. 9B and 10A-10J.) Examples include the femoral head center. Identification of the femoral head center may assist the system in determining an accuracy of bone resections. Another example feature is the femoral canal entry. Identification of the femoral canal entry may be used by the system, together with the femoral head center, to calculate and/or identify the femoral mechanical axis (e.g., the primary axis of the femur). Varus/valgus, flexion/extension, and/or rotation values (e.g., angles) of a femoral implant may be computed by the system (e.g., by the headset 102 or another computing device) relative to the femoral mechanical axis. Another example feature is the posterior condyles. Identification of the posterior condyles relative to the A/P axis may be used by the system to determine the posterior condylar axis (PCA), which may be used for the femoral rotational alignment. Other example features are the anterior and posterior trochlear groove. The anterior and posterior trochlear groove may be used by the system to determine the A/P axis (or Whiteside's Line). This axis may be used for the femoral rotational alignment. Other example features are the medial and lateral epicondyles. The medial and lateral epicondyles points may be used to determine the epicondylar axis (e.g., TEA). This axis may be used for the femoral rotational alignment. Also, the medial/lateral sizing of the femoral component may be suggested by the system based on digitized points relating to the medial and lateral epicondyles. Other example features which may be identified are the medial and lateral distal condyles, which may be relative to the femoral mechanical axis. These points may be used by the system to compute the distal femoral resection level. Another example feature which may be identified is the anterior cortex. These point(s) may be captured along the anterior cortex of the femur. The point may be used by the system for the sizing of the femur and to gauge anterior femoral notching of the implant. Using the identified femoral landmarks, such as the femoral head center, femoral canal entry, distal/posterior condyles, the system may compute the resection plane to meet a specified femoral flexion, varus/valgus angle, and/or resection depth. The femoral flexion parameter, varus/valgus angle, and/or resection depth may be provided to headset systems described herein, such as by a medical provider.

Tibial landmarking may also be performed during total knee replacement procedures described herein. During a tibial landmarking procedure, a medical provider may be prompted (e.g., using a display of the headset 102 of FIG. 1) to mark and/or otherwise indicate a variety of features related to the tibia. (See also FIGS. 10K-10T.) Examples include the malleoli. When located, these two points (medial and lateral) may allow the system to calculate the mechanical axis of the tibia. The varus/valgus, flexion/extension and rotation values may be computed by the system relative to the mechanical axis. Another example feature is the medial third of the tubercle. The tibial canal entry point may be identified as the entrance point of the intramedullary canal. Systems described herein may ensure the Anterior/Posterior (A/P) positioning of the tibial implant falls between the middle and one-third of the anterior edge of the tibial plateau. Another example feature is the tibial canal entry. The tibial canal entry point may be identified as the entry point of the intramedullary canal. Systems described herein may ensure the A/P positioning of the tibial implant falls between the middle and one-third of the anterior edge of the tibial plateau. Another example feature is the PCL insertion point. Tibial implant rotation may be defined by a point in the middle of where the native PCL attaches to the posterior tibia and one on the medial third of the tibial tuberosity. Other example features are the medial and lateral plateau resection references. The medial and lateral plateau resection references may be used by systems described herein to define resection depths for the proximal tibial resection. The depths may generally be below the points indicated by the medical professional.

Accordingly, systems described herein (e.g., using the headset 102 of FIG. 1) may prompt a medical provider to indicate any or all of the described features in preparation for making an intra-operative plan for total knee replacement. In some examples, additional features may be collected. The system may accordingly identify the position of each of the features relative to the markers, in a coordinate system defined by the markers in some examples.

Systems described herein may perform any of a variety of operations and/or guidance based on the identity and location of the collected features. In the example of a total knee operation, the identified features may be used to size the implant construct, such as to size the femoral component, size the tibial component, and/or size the tibial insert thickness. In this manner, systems described herein may provide for intra-operative computing of the implant size based on location information collected during the procedure.

Another example in the context of a total knee replacement is that systems described herein may identify and/or correct limb deformity. For example, the system may identify a center of the femoral head, identify the mechanical axis of the limb, and/or identify the deformity using location information for anatomical features indicated by the medical provider. In this manner, systems described herein may provide guidance to the medical provider to correct the deformity back to the mechanical (or other desired) axis.

Another example in the context of a total knee replacement procedure is that systems described herein may define femoral and/or tibial resection angles. Systems may define these angles, for example, based on the inputs provided in the above-described identification and/or correction of limb deformity (e.g., center of the femoral head, mechanical axis of the limb). Systems (e.g., the headset 102 of FIG. 1 and/or other computing system) may compute desired varus/valgus resection angles of the femur and/or tibia. Systems may compute the desired flexion in femoral resection and posterior slope of the tibial resection. These calculations may be made using location information obtained from medical providers using marking instruments as described herein. In some examples, systems may set a desired femoral rotation to allow for an associated resections (e.g., 4-in-1) using any one of or combination of axes including posterior condylar axis (PCA), transepicondylar axis (TEA), and/or A/P axis (e.g., Whiteside's Line).

Another example in the context of a total knee replacement procedure is that femoral and/or tibial resection depths may be defined using systems described herein. For example, systems may define a femoral component thickness, tibial component thickness, and/or tibial insert thickness. Desired tibial component position and rotation may also be defined.

In this manner, medical providers may provide location information to systems described herein by indicating particular anatomical features using a tracked pointer. The systems may use the location information to calculate various parameters relevant to a procedure—e.g., limb deformity, correction, angles, and/or depths in the example of total knee replacement. Systems may then allow for the medical provider to make real-time adjustments to resections (e.g., distal femoral, proximal tibial and posterior condylar resections) to accommodate for soft tissue (e.g., collateral ligament) behavior. Systems may then provide guidance to position a final implant construct using the location information and/or calculated parameters.

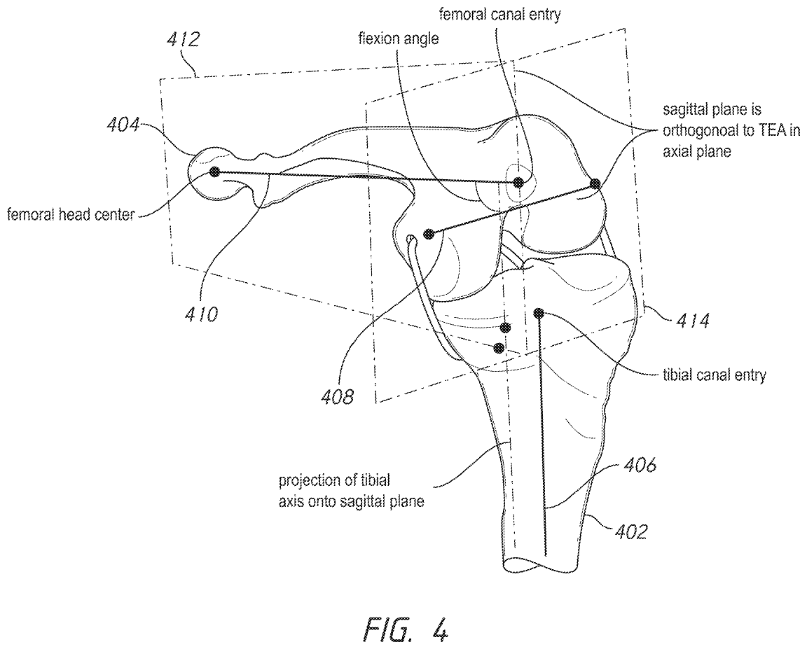

FIG. 4 is a schematic illustration of aspects of an intra-operative plan which may be generated by example systems arranged in accordance with examples described herein. The intra-operative plan may be created, for example, by the head-mounted display 202 in accordance with the executable instructions for developing intra-operative plan 214 of FIG. 2. The intra-operative plan may be created using the anatomical positions identified by the medical provider, for example, as described with respect to FIG. 3. FIG. 4 illustrates a model of the patient's knee. Identified on the model are tibia 402, femur 404, tibia mechanical axis 406, medial/lateral axis 408, femur mechanical axis 410, sagittal plane 412, and axial plane 414. These calculations may be used to guide surgical intervention, such as to guide a medical professional in placing resection guides to resect along one or more resection planes. For example, a normal to a resection plane may be computed from a cross product of the femur mechanical axis 410 rotated about the medial-lateral axis 408 by the flexion angle, and the medial-lateral axis 408 rotated about an anterior-posterior axis by the desired varus/valgus angle. The femur mechanical axis 410 may be computed from the difference between the femoral head center and the canal entry. The medial-lateral axis 408 is computed by projecting the difference between posterior condylar landmarks onto the axial plane 414. The axial plane 414 is the plane that is orthogonal to the femur mechanical axis 410. The anterior-posterior axis is computed from a cross product of the femur mechanical axis 410 and the medial-lateral axis 408. A location of a distal resection plane along the computed normal may be found by projecting distal condyle landmarks onto the computed normal and setting an offset of the resection plane equal to the average of the projections of the distal condyle landmarks. One or more headset systems or other computing systems described herein may perform the described computations.

FIG. 5 is an illustration of a medical provider placing a resection guide in accordance with surgical guidance provided in accordance with examples of systems and methods described herein. The medical provider 304 is, like in FIG. 3, wearing headset 308 and operating on a patient having the marker 322 and marker 330 placed on leg bones. Based on the intra-operative plan developed by the headset 308, for example, as described with reference to FIG. 2 and/or FIG. 4, the medical provider 304 may place a resection guide 502. The resection guide 502 may be placed to align with resection plane 414 as calculated with respect to FIG. 4.

Accordingly, the headset 308 may display the resection plane 414 aligned with the patient in the coordinate system used by the headset 308. The headset 308 may display errors in position and/or rotation of the resection guide 502 from a calculated intended position of the resection plane 414. For example, the resection guide 502 may have fiducials, such as fiducial 504, fiducial 506, fiducial 508, and fiducial 510. The fiducials may be positioned in a predetermined pattern relative to the resection guide 502, and in particular, relative to a resection plane that will be identified by a placement of the resection guide 502. In this manner, the headset 308 can compare a current position of the resection guide 502, and resulting projected resection plane, with the resection plane 414 calculated in the intra-operative plan, and suggest modifications.

A pre-operative plan may include the position and/or rotation of one or more implants (and their associated resection planes) and a set of landmarks identified on pre-operative imagery (CT, MRI, etc.). These landmarks can then be spatially registered with landmarks identified intra-operatively, thus defining the intended position and rotation of the implants.

As an example, after a resection is made, the surgeon can identify the cut plane, and the executable instructions can update the displayed metrics to reflect the actual cut, rather than the planned one. Based on those metrics, the surgeon may decide to alter the placement of subsequent resections. One example of the dynamic change of the plan is use of the measurements to determine an optimal approach for the procedure (e.g., placement of the implant, proper balancing of the position of the implant not just based on relationship of the hard tissue that is measured intra-operatively, but the impact of the surrounding soft tissues bearing on the location and movement of the hard tissue). This is also a disadvantage of the pre-operative imaging which tends to be able to plan based on a certain density of tissue (e.g., hard bone but not ligaments). So, a pre-operative plan determined by existing methods that is registered does not or cannot take into account the surrounding tissues that impact placement or planning of the targeted hard tissue.

FIG. 6 is a method 600 for resolving a pose of an instrument with the system 100, such as a pointer 108, a marker 110, a resection guide 502, or the like. The method 600 may begin in operation 602 and a depth camera receives a depth image that includes a representation of one or more fiducials relative to the headset 102. The method 600 may proceed to operation 604 and a processor, such as a processor in the headset 102, determines the depth of the fiducial relative to the headset 102. Typically, more than one fiducial may be present in the image and depth information may be determined for each. The method 600 may proceed to operation 606 and the processor may use the depth information of one or more fiducials to estimate a pose (e.g., a position and/or orientation) of a marker 110 or pointer 108 in space. The method 600 may proceed to operation 608, where the vision system 112 may receive a greyscale image of the one or more fiducials for which depth information was determined. The operations 602 and 608 may occur substantially contemporaneously such that the pose may be resolved before the surgeon moves the headset 102. The method 600 may proceed to operation 610 and the processor may use the greyscale image and the estimated pose to more accurately resolve the pose of the marker and/or pointer in 3D space than with the depth information alone. For example, the processor may use PnP techniques as previously discussed. The processor may determine a final pose of the marker or pointer for use in further calculations used in the surgical intervention, such as the locations of anatomical landmarks. The operations of the method 600 may be executed in an order other than as shown.

FIG. 7 is a schematic illustration of a method of using a headset to display content on anatomy arranged in accordance with examples described herein. The method of FIG. 7 may be performed by systems described herein (e.g., the headset 102 of FIG. 1 and/or another computing system may have software, such as executable instructions, for transforming content or other objects into various coordinate systems as described). Recall systems may utilize one or more markers attached to the patient (e.g., the marker 110 of FIG. 1). The position of the markers may be identified using the fiducials attached to the markers, and the markers may be used to define a coordinate system relative to the markers. It may be desirable to display content overlaid on anatomy as described herein. For example, the headset 102 may display one or more prompts (such as to indicate certain anatomy or to prompt a medical provider to identify a procedure to perform). The headset 102 may display one or more parameters on the anatomy (e.g., one or more planes, angles, axes, and/or other features described herein). The headset 102 may display surgical guidance information (e.g., intended resection plane, recommended corrections, etc.). Certain content may be displayed in a fixed place in the field of vision (e.g., prompts in some examples may always appear in a particular location, such as a right-hand side). However, some content (e.g., resection planes, angles, axes, etc.) may be displayed on the anatomy, and the view of the content may change as the medical provider's point of view changes. Accordingly, the system may perform a variety of transforms to ensure the content is appropriately displayed.

In some examples, a transform 702 may be used to transform landmark coordinates 708 of content to fiducial coordinates 710 of a marker coordinate system. In this manner, the system may identify the appropriate location for the content relative to markers present on the patient. Another transform 704 may be used to transform from fiducial coordinates 710 of the marker coordinate system to camera coordinates 712 of the camera coordinate system. The data including the content in the camera coordinate system may be provided to the headset and/or used by the headset to further transform the data to world coordinates 714 in the environmental coordinate system using another transform 706. The headset may provide information (e.g., using localization, such as simultaneous localization and mapping (SLAM) techniques) about the location of the headset tracked in the environmental coordinate system. This information may be used together with the data of the content in the camera coordinate system to provide the content in the environmental coordinate system. In this manner, as the headset moves, the content may continue to be rendered in an appropriate position relative to the markers.

The transforms shown and described with reference to FIG. 7 may include one or more geometric transforms, such as matrix multiplications, which may transform coordinates in six degrees of freedom in some examples (e.g., three orthogonal axes and three rotations about the axes).

For example, during landmarking of a femoral canal entry, the system may access the environmental coordinate system and a femoral coordinate system that includes the landmark coordinates 708 of femur. During landmarking of femoral points, the system may access the femoral coordinate system and the marker coordinate system that includes the fiducial coordinates 710. The marker coordinate system is based on a pointer marker. When a medical provider (e.g., a medical provider 116) has placed a tip of the pointer marker (e.g., the tip 124 of the pointer 108) at the landmark, the system may capture the landmark upon detecting that the tip of the pointer marker has been in contact with the landmark for a predetermined period. Once the landmark is captured, the medical provider may provide either an indication of acceptance of the landmark or an instruction to recapture the landmark. When the indication of acceptance has been received by the headset or computing system in communication with the headset, the femoral coordinate system and the marker coordinate system may be associated with one another based on a location of the tip of the pointer marker on a landmark in an image provided by the system. Thus, the location of the landmark may be recorded in the femoral coordinate system.

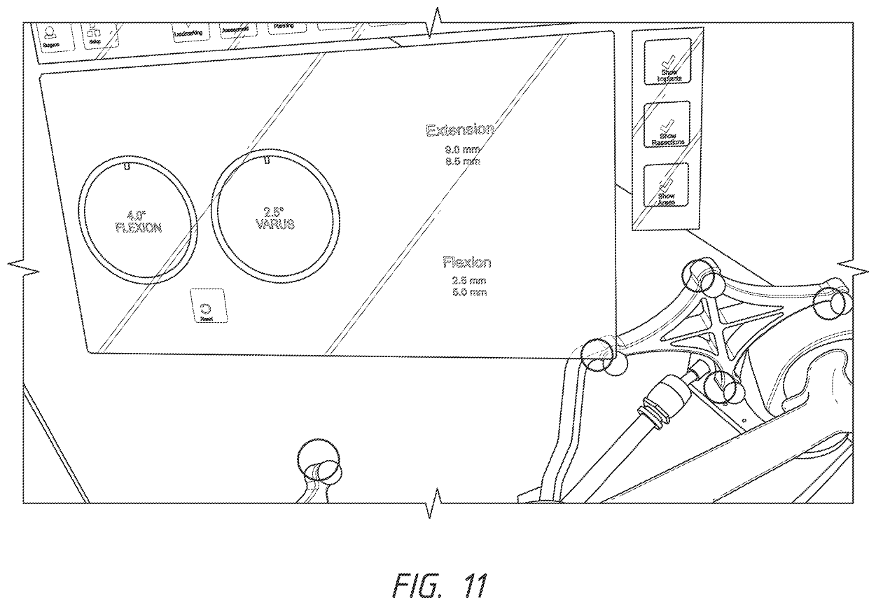

During landmarking of tibia, the system may access the marker coordinate system based on a pointer marker and a tibial coordinate system that includes the landmark coordinates 708 of tibia. During an assessment procedure (e.g., an assessment procedure with reference to FIG. 11), the system may access the tibial and femoral coordinate systems.

Accordingly, localization information obtained and/or maintained by headsets described herein may be used to ensure display of content in appropriate locations in the view of a medical provider. Localization information may advantageously be utilized in other ways in examples described herein. For example, localization information may be used when calculating parameters and/or identifying locations of anatomy when only one marker is present. Recall that a medical provider may identify anatomy in examples described herein using a pointer, and location may be determined in a marker coordinate system that may be defined by at least two markers. However, some measurements may be made using only a single marker. The femoral head center determination is an example of such a case. A single marker may be present and a joint may be rotated. To identify a center of rotation, location information provided by the headset may be used. In some examples, localization information may be used to flag erroneous anatomical identifications. For example, the localization information may provide an indication of real-world direction (e.g., up/down, left/right). If a medical provider identifies anatomy at a location that has an incorrect up/down and/or right/left relationship with other known anatomy, the system may display or otherwise record an error. For example, if the medical provider was prompted to identify the medial and lateral condyle surfaces of a right knee, the system may display or otherwise record an error if the received position information indicates that the medial and lateral condyle surfaces reported by the medical provider were reported on the wrong sides for the right knee.

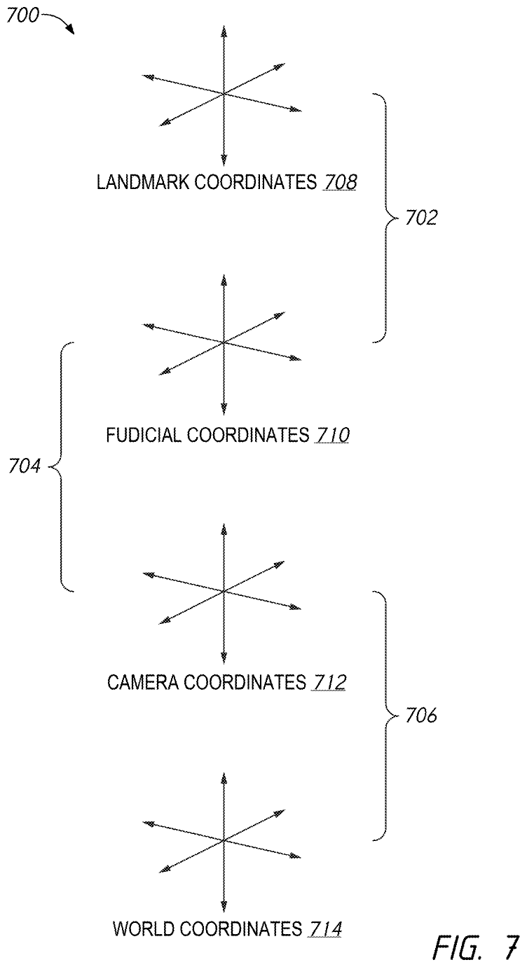

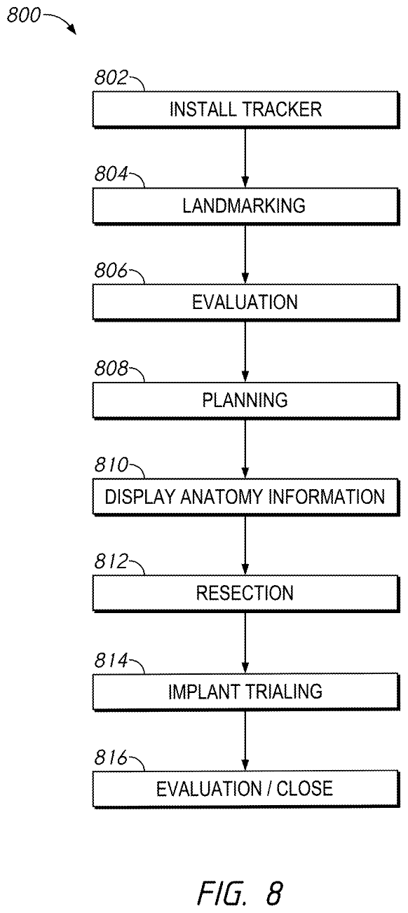

FIG. 8 is a flowchart of a method 800 of performing a surgical intervention with the system 100. The method 800 may begin in operation 802 and the surgeon makes one or more incisions in the patient's body to expose the bones for which the intervention will be performed. The surgeon may install the one or more markers 110 on the bones or other anatomy of the patient. The method 800 may proceed to operation 804 and the surgeon performs landmarking using the pointer 108, marker 110, and headset 102 to identify and track locations of the anatomical features of the patient, as previously described. The method 800 may proceed to operation 806 and the surgeon evaluates the pre-surgical condition of the anatomy. For example, in a knee replacement, the surgeon may evaluate range of motion, laxity, joint space, etc. The method 800 may proceed to operation 808 and the surgeon plans the intervention using the data determined by the system 100. For example, the system may display implant selection, implant translation or various views of the patient's anatomy. The method 800 may proceed to operation 810 and the system 100 presents information related to the anatomy of the patient. Continuing the example of a knee replacement, the system 100 may display generic representation of the femur/tibia, total component thickness, medial and lateral resection values, medial and lateral joint space values, sum of the resections and joint space on the medial and lateral sides, in flexion and in extension. The method 800 may proceed to operation and the surgeon may make the planned resection cuts. As discussed above, the surgeon may adjust the cuts based on updated intra-operative landmarks tracked by the system 100. The method 800 may proceed to operation 814 and the surgeon trials implants for fit on the patient's anatomy. For example, the surgeon may fit temporary implants to the bone to assess the size/shape of implant to be used permanently. The surgeon may fit the final implant to the anatomy. A benefit of using the systems disclosed herein may be that fewer temporary implants may be needed than with prior systems, resulting in significant cost savings. The method 800 may proceed to operation 816 and the surgeon makes a final evaluation of the anatomy and closes the surgery.

Another example of the use of a headset 102 to identify and measure anatomical landscapes is the ability to utilize the information to inform or adjust pre-operative plans or registrations made for current surgical navigation systems. The headset 102 can be used with a common set of reference fiducials shared by a surgical navigation system. The headset 102 can be used to plan intra-operatively, separately and independent of any pre-operative or additional surgical navigation system. The results of the planning and anatomical location data can be compared to the results of the pretreatment planning process. The intra-operative data can be used to correct or adjust the pretreatment plan during the procedure. This would enable static pretreatment imaging to be used for referencing anatomical locations that cannot be seen or reached by intra-operative anatomical landmark or identification. For surgical navigation systems that guide robotic interventions, the intra-operative planning system of the present disclosure can be used to independently plan and relay the planning information to the robotic system for confirmation or correction of the robotic positioning prior to the surgical intervention. In another example, the intra-operative measurement and planning systems of the present disclosure can be used as a means to track the surgical correction of the deformity post-operatively. The data acquired intra-operatively from the live anatomy and used to plan the placement of the surgical instrument can also be stored and compared to similar live post-operative data collection. For example, ankle center measurements can be repeated post-operatively and used to compare the axial alignment over time. The same type of measurements can be done pre-operatively, so that a time-based tracking of data of certain anatomical aspects can be formed and compared. The data can be used to inform progress of healing, feedback for physical therapy, or effectiveness of the procedure both short and long term. The measurements of the live anatomy pre-operatively and post-operatively can help inform and engage the patient in the recovery process by better informing them of the corrections performed intra-operatively comparatively. Current surgical navigation systems are typically comprised of a computer workstation, fixed position stereotactic cameras, markers or sensors, and an input 3D image of the patient reconstructed from an imaging source like CT, MR or X-ray.

The objective of existing navigation systems is generally to create a plan using the 3D reconstructed (e.g., pre-operative) image of the anatomy as a guide for surgery. The image is then registered to the patient's actual anatomy using fixed position stereotactic cameras, and the marker or sensor position in the coordinate system referenced to the fixed position camera. The purpose of the navigation system is to register the image to the actual anatomy so that the coordinate system can be used to guide instrumentation to the coordinates of the registered plan from the reconstructed image.

The systems and methods of the present disclosure are distinct from these systems in a number of ways. The first of which is the computer workstation and cameras are integrated into a single headset. The cameras can be monocular, time of flight, or stereotactic. In a preferred embodiment the depth camera 306 is a continuous wave time-of-flight camera. The advantage over RGB cameras is higher reliability of marker identification. RGB cameras are typically used for lower power consumption and heat generation. RGB cameras typically measure depth by measuring a size of an image (e.g., number of pixels) of a fiducial (e.g., a planar fiducial) with a known dimension and estimating a distance from the fiducial to the camera. Such systems have the disadvantages of requiring both specialized fiducials and tend to lose tracking when the fiducials are viewed at extreme angles off-normal from the planar surface of the fiducial. Another advantage of the systems of the present disclosure over existing systems is that no input 3D (e.g., pre-operative) image of the patient reconstructed from an imaging source like CT, MRI, or X-ray is needed in some examples.

In the preferred embodiment, a monocular time-of-flight camera 204 gives both depth data and infrared images used to identify the position of a particular anatomical area of interest. In a single location, the monocular camera can track poses of the markers. By a single shot, the time-of-flight camera can capture the pose of the bone marker and the pointer marker. The depth information from the time-of-flight camera is used as an initial estimate of the location of the marker. That initial estimate is then used with greyscale image data of the marker to more accurately resolve the pose of the marker (e.g., using PnP methods). The pose of the pointer may be similarly determined. The relative positions of the pointer and marker may be determined with respect to one another as previously discussed. The combination of steps, initial pose estimate and final pose calculation allow for use of a monocular time of flight and limited computing power in the headset to accurately resolve the location of anatomical points of interest. The location of the camera in the headset can then be dynamically moved so that a new point of interest can be acquired. This is a benefit over existing systems with fixed or bulky cameras that cannot easily be moved to a new point of view and do not image the surgical field from the point of view of the surgeon. Additional captures or poses add additional reference points in the coordinate system The pose of a single anatomical location is reconciled to other poses of anatomical interest to create a relative coordinate system, the known location of each anatomical area interest to each other. The ability to move the cameras while acquiring points of interest allows for expanded access to anatomical locations over fixed mounted camera systems. Furthermore, systems of the present disclosure may have a cost benefit over existing systems. For example many existing navigation systems rely on expensive, bulky fixed cameras that may cost several hundred thousand dollars. The systems of the present disclosure may cost significantly less with comparable or better performance than existing systems.

The relative coordinate system developed and utilized in the systems of the present disclosure is differentiated over existing augmented reality headsets by the way it dynamically acquires locations of anatomical interest without the need to register the locations to a prior reconstructed image from an image source like CT, MR or X-ray. In the preferred embodiment the systems of the present disclosure use a monocular time-of-flight camera to acquire both depth and infrared images of the pose of the markers. The markers used are IR markers for greater assurance that the markers can be seen in ambient light over existing QR code markers. Once enough data points are gathered and the user is satisfied, the relative coordinate system can be used to guide instruments to the anatomical point of interest specific to that instrument or procedure. The ability of the user to move positions of the headset for better access or visibility of anatomical areas of interest unlocks that capability of the system as a whole to not rely on prior imaging sources, and instead gathers as much actual anatomical locations as needed to accurately reflect the anatomy of interest.

FIG. 9A is an image of a display which may be presented to a medical provider through an augmented reality headset in accordance with examples described herein. The display shown in FIG. 9A may be used for landmarking, such as the landmarking in operation 804 of FIG. 8. Any augmented reality device may be used to display the display shown in FIG. 9A, such as the headset 102 of FIG. 1. The menu shown in the display of FIG. 9A may accordingly be superimposed over actual views of the environment (e.g., over views of a patient and/or portions of anatomy). In some examples, the menu shown may be positioned within the headset field of view in a fixed location in the field of view (e.g., in the center of the field of view), even as the medical provider moves around and the field of view of the actual scene (e.g., of the patient and the anatomy) changes. In some examples, the menu may be positioned in a fixed location in the environmental coordinate system and may be viewed when the field of view of the surgeon includes all or a portion of the location in which the menu is positioned.

The menu shown in FIG. 9A may be displayed to prompt a medical provider to identify one or more anatomical features that may be used in generating surgical guidance. Accordingly, the display may provide a list or other display of anatomical features to be identified by the medical provider. In the example of FIG. 9A, femoral landmarks and tibial landmarks are listed for the medical provider. The femoral landmarks include the femoral head center, epicondyle lateral, epicondyle medial, femur intramedullary canal, distal femur lateral, distal femur medial, posterior femur lateral, posterior femur medial, anterior cortex, anterior trochlea, and posterior trochlea. Additional, fewer, and/or different anatomical features may be used in other examples. The tibial landmarks shown in FIG. 9A include tibial tubercle, PCL insertion point, tibial canal entry, tibial sulcus lateral, tibial sulcus medial, malleolus lateral, and malleolus medial. Additional, fewer, and/or different anatomical features may be used in other examples.

The landmarks displayed on the augmented reality display may be navigated through in any of a variety of ways. In some examples, a medical provider, or another person, may state the name of the landmark that is to be identified. After speaking the name, e.g., “femoral head center,” the medical provider or other person may identify the landmark using a trackable pointer, such as the pointer 108 of FIG. 1. In this manner, a computing device, such as the headset and/or a computing system in communication with the headset, may associate the named landmark with the location specified by the pointer. The association may be stored (e.g., by storing coordinates of the identified landmark in a coordinate space defined by multiple markers). The association may be stored in memory or other electronic storage. In other examples, the medical provider or other person may perform a gesture or other action to select a landmark that will then be identified. An association between a location of the landmark and the name of the landmark may then similarly be stored. In other examples, the headset may simply highlight, provide audible output, or otherwise indicate which landmark should be identified, and the medical provider may identify the indicated landmark. In some examples, the medical provider may simply identify the landmarks in order. In any of these examples, ultimately associations are generated between the landmark and a location of the landmark as identified by the tracked pointer.

FIG. 9B is an image of a view 900 from an augmented reality headset during an example of a landmarking process arranged in accordance with examples described herein. For example, the image shown in FIG. 9B may correspond to a view of a medical provider through a headset, such as the headset 102 of FIG. 1, during a landmarking process. In the view of FIG. 9B, a femur 902 is shown. A femoral marker 904 is attached to and/or positioned proximal to the femur 902. The femoral marker 904 includes fiducials, which are shown with circles around them in the example of FIG. 9B. The circles are generated by the headset to indicate that the headset has identified the fiducial. A pointer 906 is shown in FIG. 9B. The pointer 906 includes fiducials, which are shown with circles around them in the example of FIG. 9B, such that the augmented reality headset may similarly track a position of the pointer 906 based on the fiducials. The markers, pointer, and fiducials of FIG. 9B may be implemented by and/or used to implement the system of FIG. 1, FIG. 3, and/or FIG. 5 in some examples.

During the landmarking process, a medical provider may move a tip of the pointer 906 to touch each named landmark. When the medical provider has positioned the tip of the pointer 906 at the landmark, the medical provider may provide an indication that the tip is at the landmark. The indication may be, for example, keeping the tip stationary for a threshold amount of time (e.g., 1 second, 2 seconds, 3 seconds, 4 seconds). Other indications may be a spoken indication, or other type of user interface indication, such as by pressing a button in communication with the headset and/or performing a gesture. When the indication has been received by the headset or computing system in communication with the headset, an indicator 908 may be displayed in augmented reality overlaid on the anatomy. For example, the indicator 908 may appear at a tip of the pointer 906 when the landmark is indicated by the pointer 906. In some examples, an indicator may change in appearance from when it first appears to when it is established and fixed in the scene. For example, an indicator may appear in one color (e.g., blue) when the pointer tip has been in a position for one threshold amount of time. This may provide an indication that the computing system believes the medical provider to be indicating this location for the landmark. If the pointer tip remains in that position for another threshold amount of time, the indicator may appear in another color (e.g., green) to indicate the association between the landmark and that location has been stored. The indicators may be fixed to their locations on the anatomy in the view of the medical provider, such that as the medical provider changes their field of view, the indicators remain on the landmark locations of the anatomy as viewed by the medical provider through the headset.

As the landmarks are identified, additional calculations or features that utilize the identified landmarks may be made (e.g., by the headset or a computing system in communication with the headset). When sufficient landmarks are identified to position a particular feature of interest, it may be displayed in the headset view. Furthermore, axes and planes for defining a virtual surgical space may be computed based on the identified landmarks. The anatomical landmarks, the axes and/or the planes may be used by the system for surgical planning and balancing. For example, in FIG. 9B, an axis 910 may be calculated by a computing system based on a location of multiple identified landmarks and displayed in the augmented reality view. An initial position of an implant 912 may be calculated or otherwise identified by a computing system based on identified landmarks, axes and/or planes, and may be displayed in the augmented reality view over the anatomy.

Using the landmarking process described referring to FIG. 3 and FIG. 9B, femoral landmarks may be obtained. FIG. 10A shows images of views from an augmented reality headset showing landmarks of a femur in accordance with examples described herein. For example, the femoral landmarks may include femur intramedullary canal, epicondyle lateral, epi condyle medial, posterior femur lateral, posterior femur medial, anterior trochlea, posterior trochlea, distal femur lateral, distal femur medial, anterior cortex as shown in FIG. 10A.

To obtain the femoral landmarks, a pointer 1006, such as the pointer 906, and a femoral marker 1004 on a femur 1002 in a view of a medical provider through a headset, such as the headset 102 of FIG. 1, may be used. In some embodiments, the femoral landmarks may be recorded using a coordinate system based on the femoral marker 1004. For example, as shown in FIG. 10B, the medical provider may use the pointer 1006 (e.g., the pointer 326 of FIG. 3) to have the tip of the pointer 1006 at the femoral entry point on the femur 1002 that is the deepest point of the intercondylar notch. Thus, the system may obtain the landmark of an intramedullary canal 1008.

The epicondyle medial and lateral are used to determine the epicondylar axis which is used for the femoral rotational alignment. The medial and lateral sizing of the femoral component is suggested based on these digitized points. For example, as shown in FIG. 10C, using the pointer 1006, an epicondyle lateral 1010 may be obtained.

Posterior femoral condyles are used to determine the posterior condylar axis (PCA), which is used for the femoral rotational alignment. The knee should be flexed 90 degrees before acquisition of the points. For example, as shown in FIG. 10D, using the pointer 1006, a posterior femur lateral 1012 may be obtained. FIG. 10E also shows that, using the pointer 1006, a posterior femur medial 1014 may be obtained.

Groove points of anterior and posterior trochlea may be used to determine the Anterior/Posterior (A/P) axis, which is used for the femoral rotational alignment. For example, as shown in FIG. 10F, using the pointer 1006, the groove point of an anterior trochlea 1016 may be obtained. FIG. 10G also shows that, using the pointer 1006, the groove point of a posterior trochlea 1018 may be obtained.

Distal femur lateral and medial are used to compute a level of distal resection. For example, as shown in FIG. 10H, using the pointer 1006, a distal femur lateral 1020 may be obtained. To obtain the tibial landmarks, a marker of the pointer 1006 and a tibial marker 1026 in a view of a medical provider through a headset, such as the headset 102 of FIG. 1, may be used. In some embodiments, the tibial landmarks may be recorded using a coordinate system based on the tibial marker 1026. FIG. 10L is an image of a view 1000 from an augmented reality headset during an example of a landmarking process arranged in accordance with examples described herein. For example, the image shown in FIG. 10L may correspond to a view of a medical provider through a headset, such as the headset 102 of FIG. 1, during a landmarking process. In the view of FIG. 10L, a tibia 1028 is shown. The tibial marker 1026 is attached to and/or positioned proximal to the tibia 1028. The tibial marker 1026 includes fiducials, which are shown with circles around them in the example of FIG. 10L. The circles are generated by the headset to indicate that the headset has identified the fiducial. A pointer 1006 is shown in FIG. 10L. The pointer 1006 is attached to a marker having a plurality of fiducials, such that the augmented reality headset may similarly track a position of the pointer 1006. The markers, pointer, and fiducials of FIG. 10L may be implemented by and/or used to implement the system of FIG. 1, FIG. 3, and/or FIG. 5 in some examples. For example, as shown in FIG. 10L, the medical provider may use a pointer 1006 (e.g., the pointer 326 of FIG. 3) to have the tip of the pointer 1006 on the medial third of tibial tuberosity. Thus, the system may obtain a landmark of a tibial tubercle 1030.

For example, as shown in FIG. 10M, the medical provider may use the pointer 1006 (e.g., the pointer 326 of FIG. 3) to have the tip of the pointer 1006 at an entrance point of the intramedullary canal 1008 on the tibia 1028. Thus, the system may obtain the landmark of a tibial canal entry 1032. The tibial canal entry 1032 may be centered along medial/lateral axes. As described earlier, the systems may ensure the Anterior/Posterior (A/P) positioning of a tibial implant falls between the middle and one-third of the anterior edge of the tibial plateau.

For example, as shown in FIG. 10N, the medical provider may use the pointer 1006 (e.g., the pointer 326 of FIG. 3) to have the tip of the pointer 1006 at an area of PCL insertion 1034 on the tibia 1028. Thus, the system may obtain the landmark of the area of PCL insertion 1034. The neutral rotation is defined by a point in the middle of the PCL insertion area on the tibial plateau and one on the medial third of the tibial tuberosity. This axis may lie perpendicular to the posterior edges of the proximal tibia.

Tibial sulcus medial and lateral may be used as medial and lateral plateau resection references to compute a level of resection. For example, as shown in FIG. 10O, using the pointer 1006, a tibial sulcus medial 1036 may be obtained to represent a lowest point of medial tibial plateau. FIG. 10P also shows that, using the pointer 1006, a tibial sulcus lateral 1038 may be obtained to represent a lowest point of lateral tibial plateau. A planned tibial proximal resection plane may be set below the obtained tibial sulcus medial 1036 and tibial sulcus lateral 1038.

To compute the mechanical axis of the tibia 1028, medial and lateral malleoli may be used. For example, as shown in FIGS. 10Q and 10R, using the pointer 1006, a lateral malleolus 1040 on the tibia 1028 may be obtained. FIGS. 10S and 10T also show that, using the pointer 1006, a medial malleolus 1042 on the tibia 1028 may be obtained. The varus/valgus and slope values are computed relative to the mechanical axis.

As described above, landmarks on the femur 1002 and the tibia 1028 may be obtained by using the pointer 1006 to address the landmarks, while the pointer 1006 and either the femoral marker 1004 or the tibial marker 1026 may be in the view of the headset. Once the femoral and tibial landmarks have been obtained, the medical provider may proceed to the assessment mode.

FIG. 11 is an image of a view from an augmented reality headset of an evaluation interface depicting parameters relevant for a knee implant in extension and flexion. In an example where a total knee replacement procedure is performed, an initial position of both a tibial component and a femoral component of the knee implant may be calculated based on the landmarks. The initial position may be displayed using the headset such that the tibial component and the femoral component are depicted on the appropriate tibial and femoral anatomy. A medical provider may then select (e.g., using a user interface to the headset) an assessment procedure. During the assessment procedure, the patient's leg may be moved from flexion into extension (or vice versa), and relevant implant parameters may be calculated in these poses based on the locations of the landmarks. For example, medial and lateral gap distances between the femoral and tibial components may be calculated in both extension and flexion. These parameters may be displayed, as shown in FIG. 11.

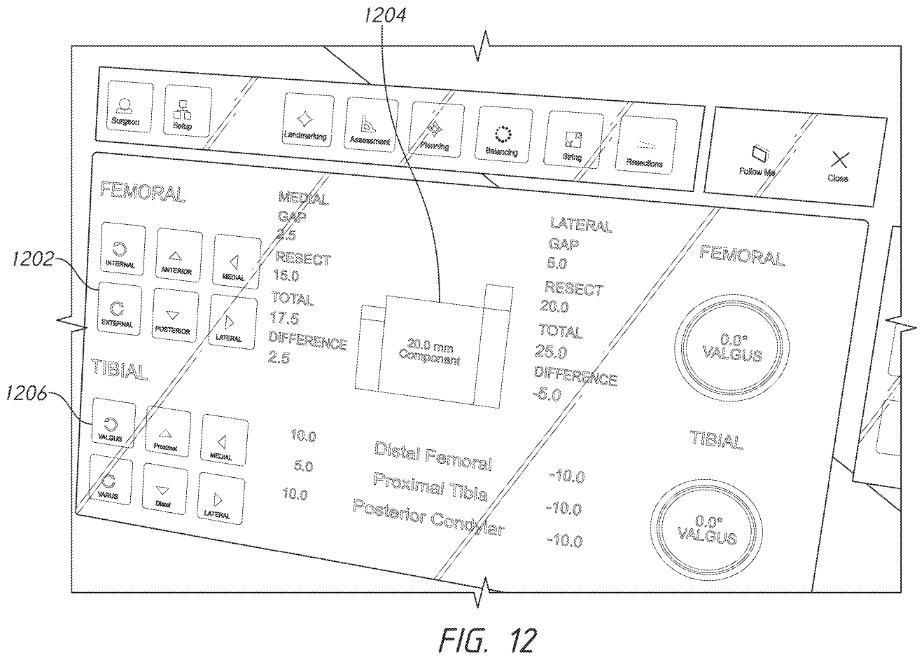

FIG. 12 is an image of a view from an augmented reality headset of a balancing interface allowing for adjustment of a knee implant in accordance with examples described herein. The image may be created by an augmented reality headset described herein, such as by the headset 102 of FIG. 1. The balancing interface allows a medical provider to make adjustments to a planned placement of a femoral and tibial component of a knee implant. For example, adjustment selectors 1202 allow a medical provider to select, for a femoral component, internal rotation, external rotation, anterior movement, posterior movement, medial movement, and/or lateral movement. Responsive to selecting any of the adjustment selectors 1202, the system may rotate and/or move a position of the femoral component. Similarly, adjustment selectors 1206 allow a medical provider to select, for a tibial component, internal rotation, external rotation, anterior movement, posterior movement, medial movement, and/or lateral movement. Responsive to selecting any of the adjustment selectors 1206 (e.g., using a gesture, such as a point), the system may rotate and/or move a position of the tibial component. A medical provider's view of the virtual tibial and femoral components displayed on the anatomy may be updated in accordance with the adjustments.

In some examples, a guidance visualization 1204 may be provided to guide a medical provider in adjusting the position of the tibial and/or femoral components. The guidance visualization 1204 shown in FIG. 12 includes a bar on either side of a square. The height of the bar may be determined by a total gap distance on the medial and lateral sides of the implant. So, the height of the bar on one side of guidance visualization 1204 may be based on a medial gap distance (which may include a sum of a gap and a resection depth). The height of the bar on the other side of the guidance visualization 1204 may be based on a lateral gap distance (which may include a sum of a gap and a resection depth). A medical provider may make adjustments to the position of the tibial and femoral components using the adjustment selectors 1202 and adjustment selectors 1206 in order to reduce or eliminate a disparity between the heights of the bars—e.g., in order to reduce or eliminate a total depth of gap plus resection depth.

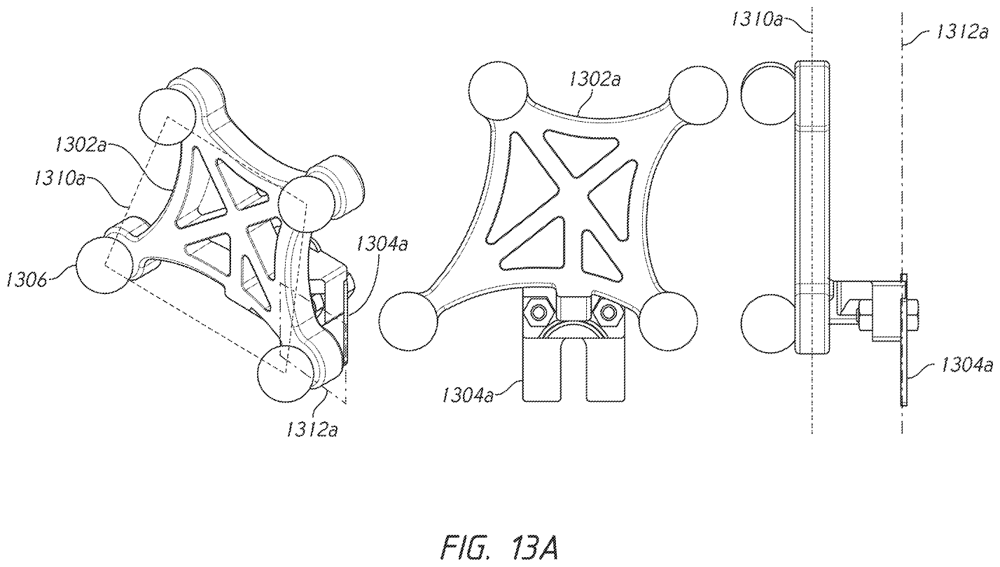

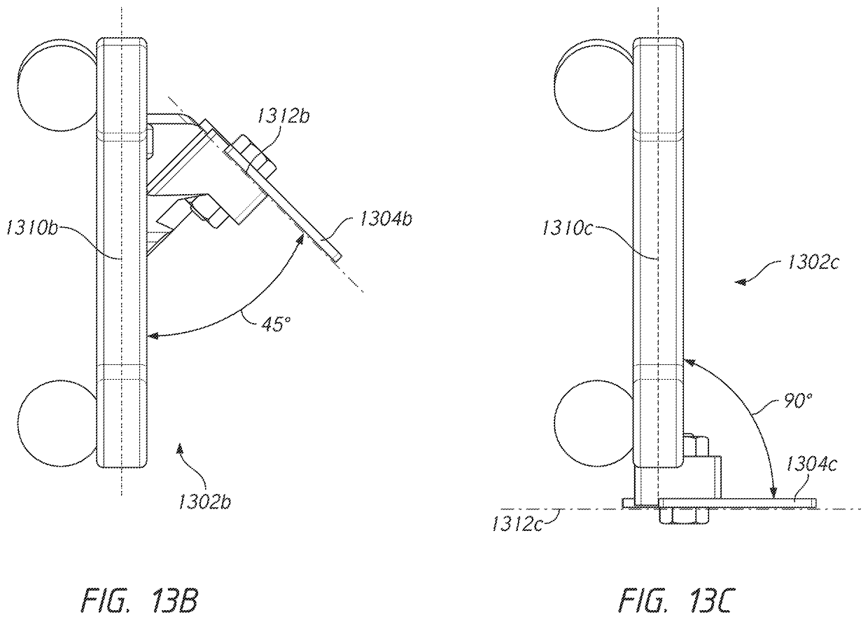

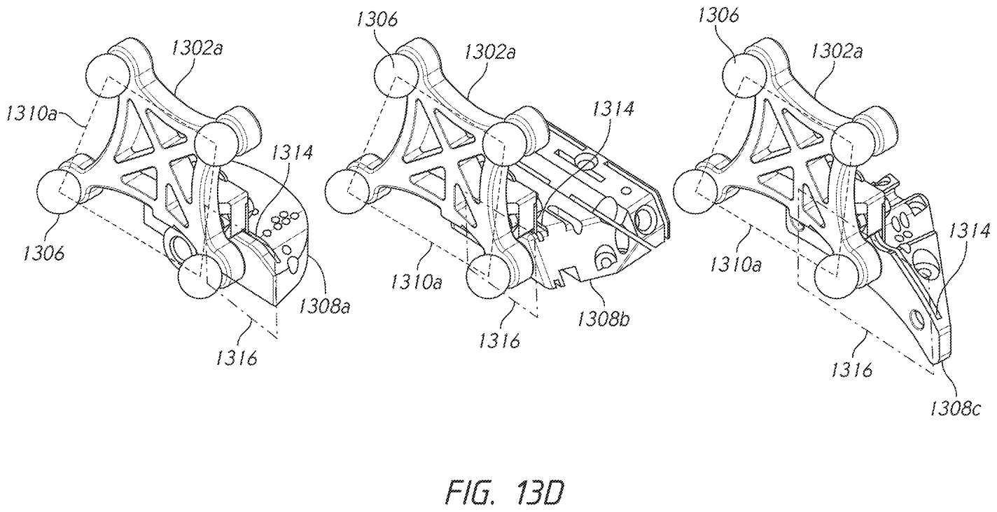

FIG. 13A shows several views of a resection marker 1302a for tracking a resection guide. The resection marker 1302a may be removably attached into the resection guide. The resection marker 1302a includes four fiducials 1306, which may be positioned in a predetermined pattern. Any number or arrangement of fiducials may be used, including the fiducials 1306 described herein. The resection marker 1302a includes an insertion member 1304a. The insertion member 1304a is shaped such that it fits into a slot of a resection guide (e.g., a slot 1314 of a resection guide 1308a-1308c in FIG. 13D). The insertion member 1304a includes a rigid portion having a width, thickness and depth selected to be inserted into a slot of resection guide. In some embodiments, the rigid portion may be substantially flat and thin. The rigid portion may have surfaces along an insertion plane 1312a (e.g., insertion plane 1312a-1312c of FIG. 13D) that corresponds to resection surfaces of a cutting device when placed in the slot of the resection guide. The thickness of the rigid portion may be slightly less than the width of the slot in a manner the insertion member 1304a may stably fit in the slot. Thus, the resection marker 1302a may be used to represent a location and orientation of the surfaces of the rigid portion approximating the resection surfaces.

In some embodiments, the insertion plane 1312a perpendicular to the depth of the rigid portion and a fiducial plane 1310a including the fiducials 1306 may be configured to be in parallel.

FIG. 13B shows a view of a resection marker 1302b for tracking a resection guide. In some embodiments, an angle between a fiducial plane 1310b including fiducials and an insertion plane 1312b along an insertion member 1304b may be 45°. Similarly to the insertion member 1304a, the insertion member 1304b is shaped such that it fits into a slot of a resection guide (e.g., a slot 1314 of a resection guide 1308a-1308c in FIG. 13D). FIG. 13C shows a view of a resection marker 1302c or tracking a resection guide. In some embodiments, an angle between a fiducial plane 1310c including fiducials and an insertion plane 1312c along an insertion member 1304c may be a right angle (90°). Similarly to the insertion member 1304a or 1304b, the insertion member 1304c is shaped such that it fits into a slot of a resection guide (e.g., a slot 1314 of a resection guide 1308a-1308c in FIG. 13D).

FIG. 13D shows several views of the resection marker 1302a attached to a resection guide 1308a, a resection guide 1308b, or a resection guide 1308c. Each of the resection guides 1308a, 1308b and 1308c may be a resection guide attached to either a femur, a tibia, etc. In some embodiments, one of the resection guides 1308a, 1308b and 1308c may be attached to a femur, and one of the others of the resection guides 1308a, 1308b and 1308c may be attached to a tibia. In some embodiments, there may be resection guides designed to be attached to specific sides of the tibia. Each of the resection guides 1308a, 1308b and 1308c includes a slot 1314 used to guide a cutting device along a resection plane 1316 during resection. The slot 1314 may define a plane where the resection is to be performed. The resection marker 1302a may be securely attached to the resection guide when the rigid portion of the insertion member 1304a is inserted into the slot 1314 of any of the resection guides 1308a, 1308b and 1308c. In this manner, by inserting the insertion member 1304a into the slot 1314 of the resection guide 1308a, 1308b or 1308c, the resection marker 1302a may be removably connected to the resection guide 1308a, 1308b or 1308c. Note that the resection marker 1302a may generally be removably connected to any of a variety of resection guides—it is not specific to a particular resection guide. In some embodiments, each slot 1314 of the resection guides, such as the resection guides 1308a, 1308b and 1308c, has a width that is slightly greater than a thickness of the insertion member 1304a. Thus, the same insertion member 1304a may be used to define any resection plane of the resection guides 1308a, 1308b and 1308c. Note also that, because the resection marker 1302a is inserted into the slot 1314 used to guide a cutting device during resection, the angle of the resection marker 1302a (e.g., an angle of the insertion plane 1312a parallel to the fiducial plane 1310a) may be representative of an angle of the resection plane 1316 of the cut to be guided by the resection guide 1308a.

Similarly, the insertion marker 1302b of FIG. 13B or the insertion marker 1302c of FIG. 13C may be securely attached to the resection guide when the rigid portion of the insertion member 1304b or 1304c is inserted into the slot 1314 of any of the resection guides 1308a, 1308b and 1308c. In this manner, by inserting the insertion member 1304b or 1304c into the slot 1314 of the resection guide 1308a, 1308b or 1308c, the resection marker 1302b or 1302c may be removably connected to the resection guide 1308a, 1308b or 1308c.

In some examples, as previously described with regard to FIG. 7, landmark coordinates of the resection guides 1308a, 1308b and 1308c based on a marker, such as a femoral marker or a tibial marker, may be obtained when the resection marker 1302a is attached to the resection guide 1308a, 1308b or 1308c by inserting the insertion member 1304a into the slot 1314 of the resection guide 1308a. In this manner, the system may identify the appropriate location of the resection plane 1316 relative to the femoral marker or the tibial marker. After identifying the resection plane 1316, the resection marker 1302a may be safely removed. Thus the resection marker 1302a may not obstruct the view surrounding the resection plane 1316. For example, an estimated resection plane based on the position of the resection marker 1302a may be displayed using an AR/VR system and/or display screen, even after the resection marker 1302a is removed from the resection guide.

A medical provider may plan the location of the resection plane 1316 by adjusting each of a pair of angles and a depth of the resection plane 1316 through an image of a view from an augmented reality headset. For example, during planning of distal resection, the system may access a marker coordinate system that includes fiducial coordinates of the resection marker 1302a and a femoral coordinate system that includes landmark coordinates (e.g., the landmark coordinates 708) of a femur; thus the system recognizes the location of the resection plane 1316 relative to the femoral marker. During planning of resections (e.g., 4-in-1) using any one of or combination of axes including posterior condylar axis (PCA), transepicondylar axis (TEA), and/or A/P axis (e.g., Whiteside's Line), the system may access a marker coordinate system that includes fiducial coordinates of the resection marker 1302a and a femoral coordinate system that includes landmark coordinates (e.g., the landmark coordinates 708) of a femur; thus the system recognizes the location of the resection plane 1316 relative to the femoral marker. During planning of proximal resection, the system may access a marker coordinate system that includes fiducial coordinates of the resection marker 1302a and a tibial coordinate system that includes landmark coordinates (e.g., the landmark coordinates 708) of tibia; thus the system recognizes the location of the resection plane 1316 relative to the tibial marker.

Once the resection planning is complete, the medical provider may be navigated to position the resection guide (e.g., the resection guide 1308a, 1308b, or 1308c) to match the planned resection plane 1316. After placing the resection guide and the resection marker 1302a is removed, resection may be performed along the slot 1314.

Accordingly, localization information obtained and/or maintained by headsets described herein based on the resection guide 1308a attached to a body part, the resection marker 1302a attached to the resection guide 1308a by having the insertion member 1304a in the slot 1314, and the landmarks of the body part, such as landmark coordinates of a femur or tibia, may be used to ensure display of the resection plane 1316 relative to the body part in appropriate locations in the view of a medical provider, without obstructing the view by the fiducials 1306 after removal of the resection marker 1302a.

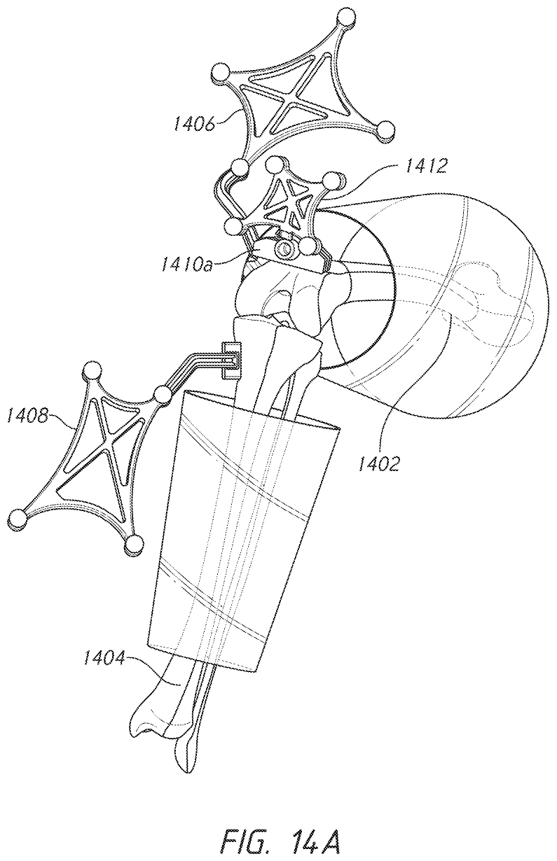

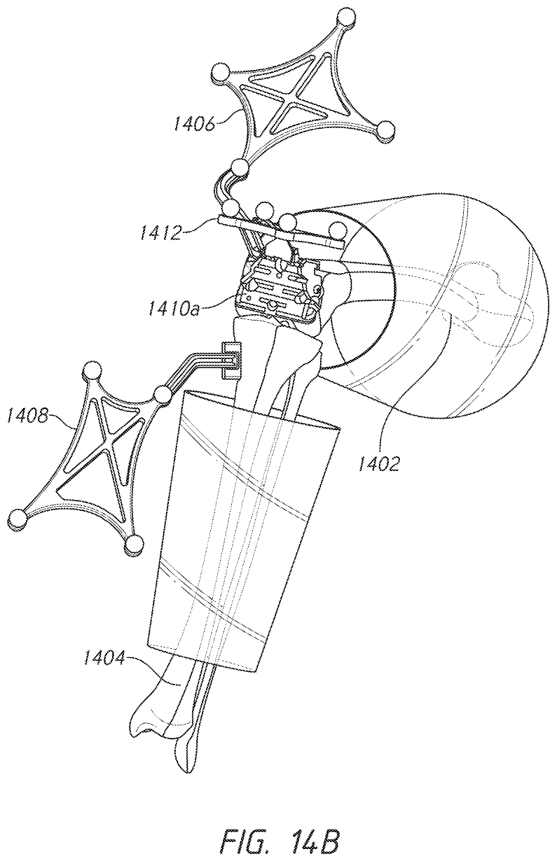

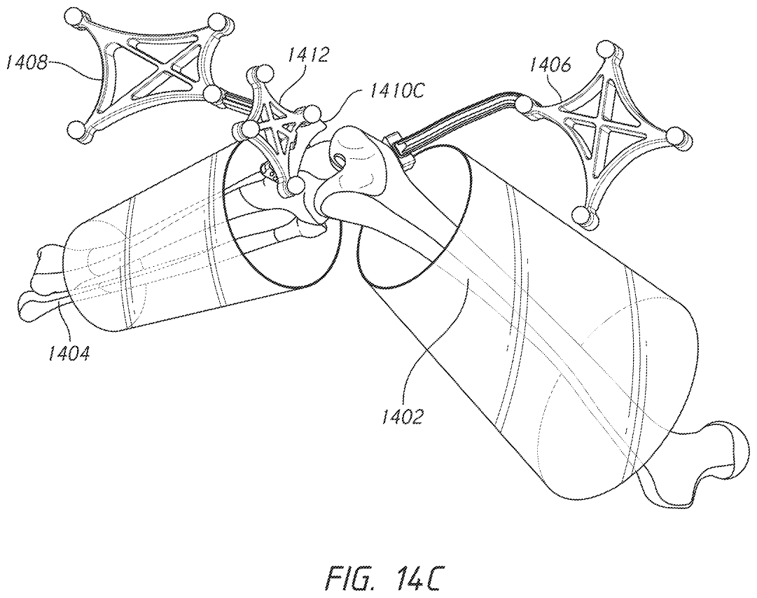

FIG. 14A is a schematic illustration of a femur 1402 and tibia 1404 having markers 1406 and 1408 and a resection guide 1410a reversibly coupled to a marker arranged in accordance with examples described herein. FIG. 14A depicts the femur 1402 and tibia 1404 in preparation for a total knee replacement procedure. A marker 1406 may be affixed or otherwise placed proximate the femur 1402. Another marker 1408 may be affixed to or otherwise placed proximate the tibia 1404. As described herein the marker 1406 may include a pattern of fiducials, and the marker 1408 may include a pattern of fiducials. The pattern of fiducials on the marker 1406 and marker 1408 may be used to define a coordinate system to determine a relative location of objects, such as one or more pointers, resection guides, anatomical features, and/or parameters relevant to a surgical procedure such as a resection. In some embodiment, a resection guide 1410a may be the resection guide 1308a of FIG. 13D placed on the femur 1402. A marker 1412 may be attached to the resection guide 1410a. For example, the marker 1412 may be removably attached to the resection guide 1410a. The marker 1412 may be implemented using the marker 1302a of FIG. 13A and may have an insertion member adapted for insertion into a slot of the resection guide 1410a. The slot may be the same slot used to guide the cutting device during resection. Examples of systems described herein may be used to guide positioning of the resection guide 1410a on the femur 1402.

FIG. 14B is a schematic illustration of a femur 1402 and tibia 1404 having markers 1406 and 1408 and a resection guide 1410b reversibly coupled to a marker arranged in accordance with examples described herein. FIG. 14B depicts the femur 1402 and tibia 1404 in preparation for a total knee replacement procedure. A marker 1406 may be affixed or otherwise placed proximate the femur 1402. Another marker 1408 may be affixed to or otherwise placed proximate the tibia 1404. As described herein the marker 1406 may include a pattern of fiducials, and the marker 1408 may include a pattern of fiducials. The pattern of fiducials on the marker 1406 and marker 1408 may be used to define a coordinate system to determine a relative location of objects, such as one or more pointers, resection guides, anatomical features, and/or parameters relevant to a surgical procedure such as a resection. In some embodiment, a resection guide 1410b may be the resection guide 1308b of FIG. 13D placed across the femur 1402 and the tibia 1404. A marker 1412 may be attached to the resection guide 1410b. For example, the marker 1412 may be removably attached to the resection guide 1410b. The marker 1412 may be implemented using the marker 1302a of FIG. 13A and may have an insertion member adapted for insertion into a slot of the resection guide 1410b. The slot may be the same slot used to guide the cutting device during resection. Examples of systems described herein may be used to guide positioning of the resection guide 1410b on the femur 1402.

FIG. 14C is a schematic illustration of a femur 1402 and tibia 1404 having markers 1406 and 1408 and a resection guide 1410c reversibly coupled to a marker arranged in accordance with examples described herein. FIG. 14C depicts the femur 1402 and tibia 1404 in preparation for a total knee replacement procedure. A marker 1406 may be affixed or otherwise placed proximate the femur 1402. Another marker 1408 may be affixed to or otherwise placed proximate the tibia 1404. As described herein the marker 1406 may include a pattern of fiducials, and the marker 1408 may include a pattern of fiducials. The pattern of fiducials on the marker 1406 and marker 1408 may be used to define a coordinate system to determine a relative location of objects, such as one or more pointers, resection guides, anatomical features, and/or parameters relevant to a surgical procedure such as a resection. In some embodiment, a resection guide 1410c may be the resection guide 1308c of FIG. 13D placed on the tibia 1404. A marker 1412 may be attached to the resection guide 1410c. For example, the marker 1412 may be removably attached to the resection guide 1410c. The marker 1412 may be implemented using the marker 1302a of FIG. 13A and may have an insertion member adapted for insertion into a slot of the resection guide 1410c. The slot may be the same slot used to guide the cutting device during resection. Examples of systems described herein may be used to guide positioning of the resection guide 1410c on the femur 1402.

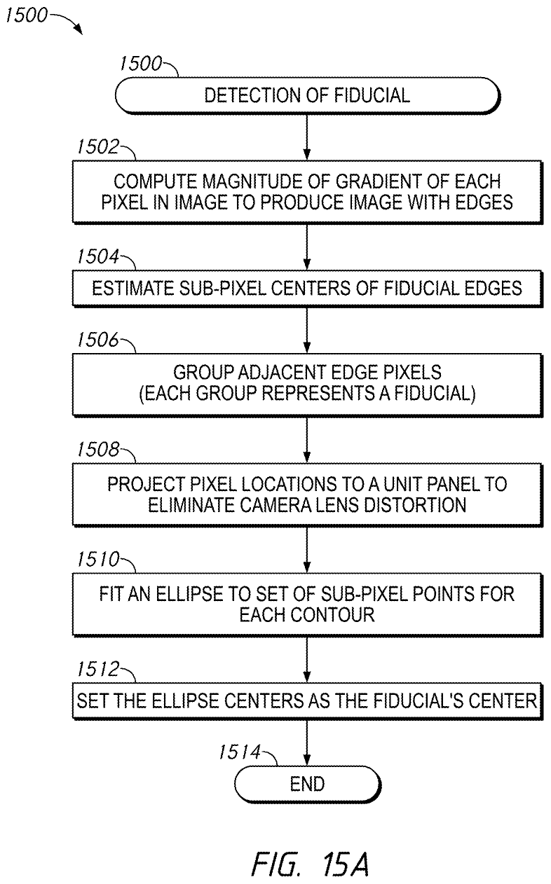

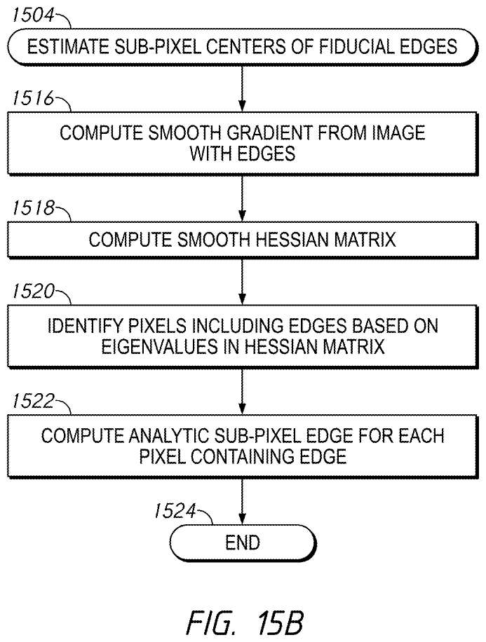

In order to identify an arrangement (e.g., a plane) including fiducials of a marker, locations of fiducials of the marker may be detected. FIG. 15A is a flow chart of a method 1500 of detecting fiducials of a marker of the system of FIG. 1. A vision system, such as the vision system 112 of FIG. 1, may capture a frame, and an image including at least a portion of a field of view, such as the field of view 120, may be obtained as the image. The image may include a marker having fiducials. The headset 102 may perform edge detection of fiducials to identify the locations of centers of fiducials. In some embodiments, the method 1500 may begin in operation 1502, and the headset 102 may compute a magnitude of a gradient of each pixel in the image based on the image. In some embodiments, the gradient of the image may be computed using Sobel filter; however, any other types of edge detection may be used. The headset 102 may produce a fiducial image including edges of the fiducials based on the magnitudes of the gradients of pixels in the image. The edges of the fiducials may correspond to boundaries of the fiducials in the image. The method 1500 may proceed to operation 1504, and the headset 102 may estimate sub-pixel centers of the edges of the fiducials based on the fiducial image. The method 1500 may proceed to operation 1506, and the headset 102 may group adjacent edge pixels in the fiducial image. Here, each group may represent a corresponding fiducial. Once one or more groups of pixels are identified, the method 1500 may proceed to operation 1508, and the headset 102 may project the pixel locations of the one or more groups of pixels to a unit plane. Here, an effect of camera lens distortion on the fiducial image due to one or more cameras in the vision system 112 may be removed. The method 1500 may proceed to operation 1510, and the headset 102 may fit an ellipse to a set of sub-pixel points that is the group of pixels. Then the method 1500 may proceed to operation 1512, and the headset 102 may take a center of the ellipse as a center of a fiducial represented by the group of pixels. By performing the method 1500 for each fiducial, the locations of the fiducials of each marker may be obtained.

FIG. 15B is a flow chart of a method 1504 of estimating sub-pixel centers of fiducial edges of FIG. 15A. In some embodiments, the method 1504 may begin in operation 1516, and the headset 102 may compute a smooth gradient of the fiducial image, with a first-derivative gaussian kernel. The method 1504 may proceed to operation 1518 and the headset 102 may compute a smooth hessian matrix using a second-derivative gaussian kernel. The method 1504 may proceed to operation 1520 and the headset 104 may identify pixels containing edges using eigenvalues in the hessian matrix. The headset 102 may detect pixels having a large negative eigenvalue as edge pixels. The method 1504 may proceed to operation 1522 and the headset 102 may compute the analytic sub-pixel edge for each edge pixel. For example, the analytic sub-pixel edge for each edge pixel may be computed using a second order Taylor series approximation computed from the first and second derivatives.

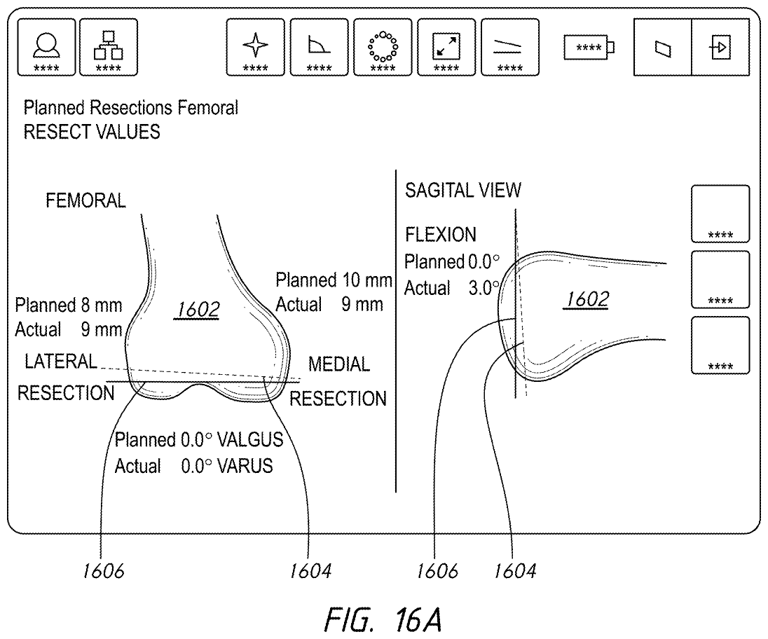

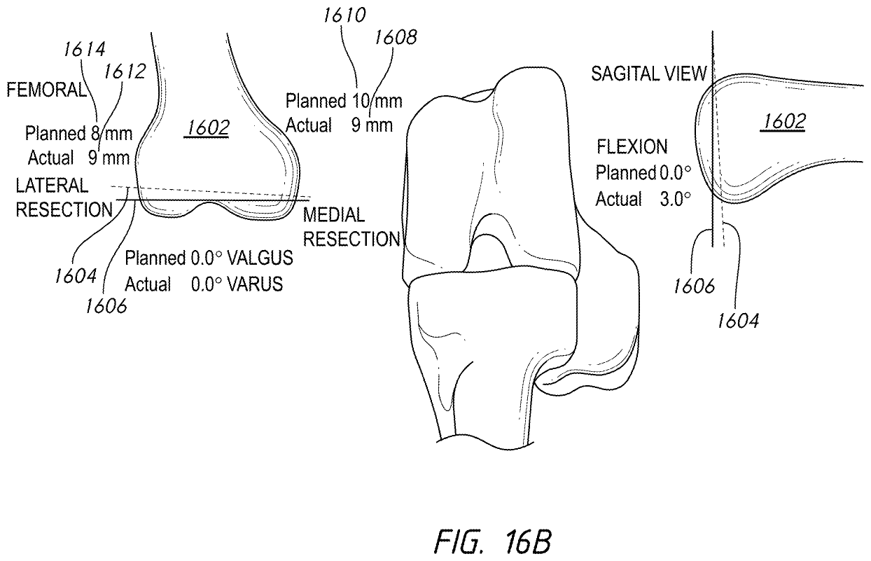

FIGS. 16A-19B are images of views from an augmented reality headset of a resection interface allowing for adjustment of a resection guide to match a planned resection plane. The image may be generated by an augmented reality headset, examples of which are described herein, such as headset 102 of FIG. 1. A resection guide, such as a resection guide 1308a, 1308b, 1308c of FIG. 13D and/or resection guide 1410 of FIG. 14A may be placed on or proximate the bone to guide a resection cut. Examples of systems described herein provide guidance for placement of the resection guide.

For example, FIG. 16A is a view illustrating a schematic view of a femur 1602 from two directions—showing a planned resection plane 1604 (shown in a blue dotted line) and an actual resection plane 1606 (shown in a white dotted line). FIG. 16B is an image of a view from the augmented reality headset of the resection interface overlaid on an actual image of the femur 1602. Note that the depictions of the femur 1602 may be schematic only in some examples—it need not be a true depiction of the patient's anatomy, just a schematic of a femur to aid in guiding resection placement. The planned resection lines may be based on the resection depths and measurements that were obtained, for example, using the balancing interface of FIG. 12. Femoral landmarks may have been obtained using a marker of the pointer 1006 and a femoral marker 1004 in a view of a medical provider through a headset, such as the headset 102 of FIG. 1. A coordinate system for representing the landmarks of the femur 1602 may be based on a femoral marker, such as the femoral marker 1004. Similarly, items, such as a resection guide and the planes 1604 and 1606, in the view of a medical provider through a headset, may be represented in the coordinate system based on the femoral marker.

A resection guide may be provided with a marker, such as a resection marker 1302a having fiducials 1306. For example, the insertion member 1304a of the resection marker 1302a of FIG. 13A may be inserted into a slot 1314 of a resection guide, such as the resection guide 1308a, 1308b or 1308c. The resection guide for a femur 1602 may be placed on or proximate the femur 1602. The augmented reality headset may recognize the fiducials of the resection marker removably coupled to the resection guide and may use the position (e.g., location and orientation) of the fiducials to generate actual resection plane information—such as actual resection plane 1606. The medical provider may manipulate and adjust a position of the resection guide, such by manually rotating and/or moving the resection guide to calibrate the position (e.g., location and orientation) of the actual resection plane 1606. In some examples, a pin, a jig or other mechanical structure may be used to aid in precision movement (e.g., less than 1 mm) of the resection guide. Adjustments can be made to resection depth, flexion/extension and varus/valgus. The medical provider may move the resection guide while observing the resection interface of FIG. 16B to observe changes in an actual medial resection 1608 and actual lateral resection 1612. By observing two views presented together, the medical provider may move the resection guide to reduce and/or minimize a distance between the planned resection plane 1604 and the actual resection plane 1606 at posterior, anterior, lateral and medial sides. For example, the medical provider may move the resection guide to reduce and/or minimize a distance between a planned medial resection 1610 and the actual medial resection 1608 and/or between a planned lateral resection 1614 and the actual lateral resection 1612. In this manner, the resection interface may guide the medical provider in accurate placement of the resection guide. Once the resection guide is positioned accurately, the medical provider may affix it to the bone and perform the resection.

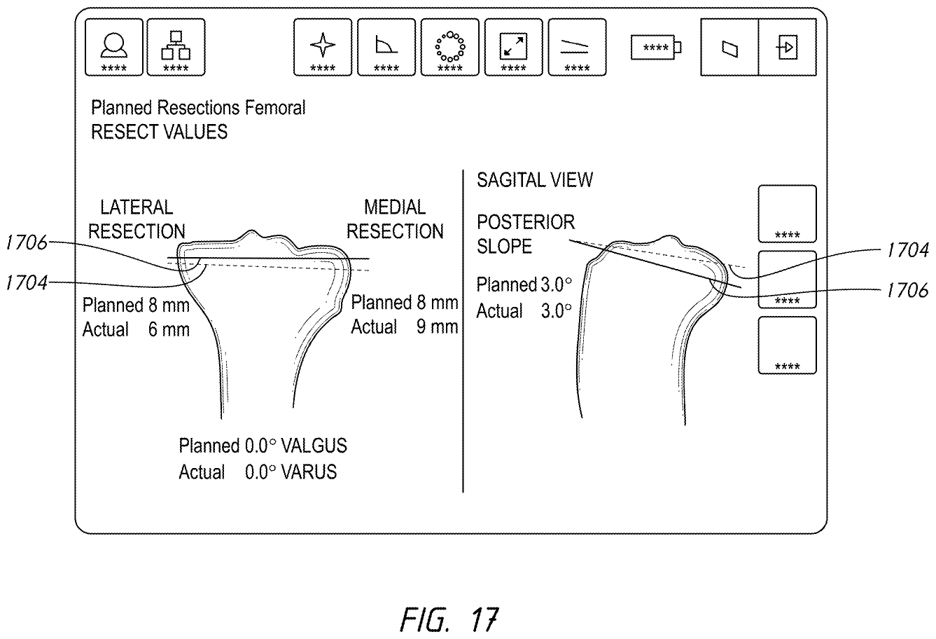

In another example, FIG. 17 is a view illustrating a schematic view of a tibia 1702 from two directions—showing a planned resection plane 1704 (shown in a dotted line) and an actual resection plane 1706 (shown in a solid line). Note that the depictions of the tibia 1702 may be schematic only in some examples—it need not be a true depiction of the patient's anatomy, just a schematic of a tibia to aid in guiding resection placement. The planned resection lines may be based on the resection depths and measurements that were obtained, for example, using the balancing interface of FIG. 12. Tibial landmarks may have been obtained using a marker of the pointer 1006 and a tibial marker 1026 in a view of a medical provider through a headset, such as the headset 102 of FIG. 1. A coordinate system for representing the landmarks of the tibia 1702 may be based on a tibial marker, such as the tibial marker 1026. Similarly, items, such as a resection guide and the planes 1704 and 1706, in the view of a medical provider through a headset, may be represented in the coordinate system based on the tibial marker.

A resection guide may be provided with a marker, such as a resection marker 1302a having fiducials 1306. For example, the insertion member 1304a of the resection marker 1302a of FIG. 13A may be inserted into a slot 1314 of a resection guide, such as the resection guide 1308a, 1308b or 1308c. The resection guide for a tibia 1028 may be placed on or proximate the tibia 1702. The augmented reality headset may recognize the fiducials of the resection marker removably coupled to the resection guide and may use the location and orientation of the fiducials to generate actual resection plane information—such as the actual resection plane 1706. The medical provider may manipulate and adjust a position of the resection guide, such by manually rotating and/or moving the resection guide. In some examples, a pin, a jig or other mechanical structure may be used to aid in precision movement (e.g., less than 1 mm) of the resection guide. Adjustments can be made to the resection depth, posterior tibial slope and varus/valgus. The medical provider may move the resection guide while observing the resection interface of FIG. 17 to observe changes in the actual resection plane 1706. By observing two views presented together, the medical provider may move the resection guide to reduce and/or minimize a distance between the planned resection plane 1704 and the actual resection plane 1706 at posterior, anterior, lateral and medial sides. In this manner, the resection interface may guide the medical provider in accurate placement of the resection guide. Once the resection guide is positioned accurately, the medical provider may affix it to the tibia, remove the resection marker and perform the resection.

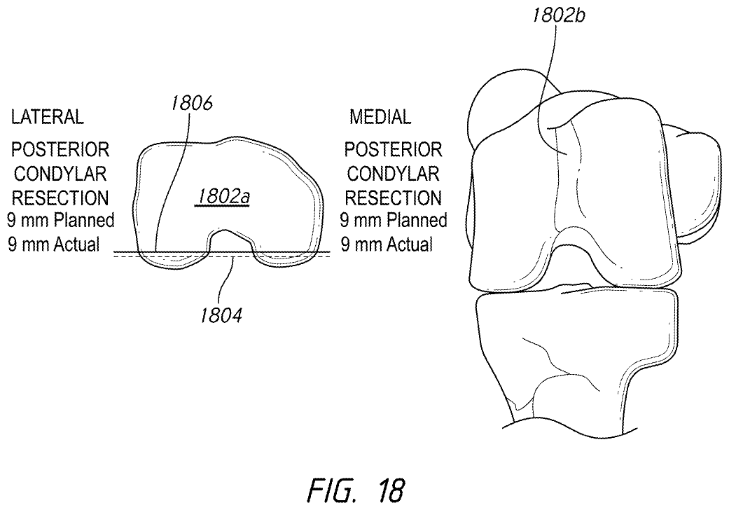

After the distal femoral, or tibial resection, a chamfer cut may be made around the resection surface of the femur or tibia to smooth edges. To perform the chamfer cut, 4-in-1 resection may be performed. For example, FIG. 18 is a view illustrating a schematic view of a femur 1802a during femoral 4-in-1 resection showing a planned posterior condylar resection plane 1804 (shown in a blue dotted line) and an actual posterior condylar resection plane 1806 (shown in a white dotted line) overlaid on an actual image of the femur 1802a. Note that the depictions of the femur 1802a may be schematic only in some examples—it need not be a true depiction of the patient's anatomy, just a schematic of a femur to aid in guiding resection placement. The planned resection lines may be based on the resection depths and measurements that were obtained, for example, using the balancing interface of FIG. 12. Femoral landmarks may have been obtained using a marker of the pointer 1006 and a femoral marker 1004 in a view of a medical provider through a headset, such as the headset 102 of FIG. 1. A coordinate system for representing the landmarks of the femur 1602 may be based on a femoral marker, such as the femoral marker 1004. Similarly, items, such as a resection guide and the planes 1804 and 1806, in the view of a medical provider through a headset, may be represented in the coordinate system based on the femoral marker.

In some embodiments, a resection guide may be specific to a knee implant system to be used. The resection guide may be provided with a marker, such as a resection marker 1302a having fiducials 1306. For example, the insertion member 1304a of the resection marker 1302a of FIG. 13A may be inserted into a slot 1314 of a resection guide, such as the resection guide 1308a, 1308b or 1308c. The resection guide for the femoral 4-in-1 resection with the resection marker 1302a may be placed on the resected distal femoral surface. The augmented reality headset may recognize the fiducials of the resection marker removably coupled to the resection guide and may use the location and orientation of the fiducials to generate actual resection plane information—such as the actual posterior condylar resection plane 1806. The medical provider may manipulate and adjust a position of the resection guide, such by manually rotating and/or moving the resection guide. In some examples, a pin, a jig or other mechanical structure may be used to aid in precision movement (e.g., less than 1 mm) of the resection guide. Adjustments can be made to resection depth and rotation manually. The medical provider may move the resection guide while observing the resection interface of FIG. 18 to observe changes in the actual posterior condylar resection plane 1806. By observing the view, the medical provider may move the resection guide to reduce and/or minimize a distance between the planned posteri or condylar resection plane 1804 and the actual posterior condylar resection plane 1806 at posterior, anterior, lateral and medial sides. In this manner, the resection interface may guide the medical provider in accurate placement of the resection guide. Once the resection guide is positioned accurately, the medical provider may affix it to the femur, remove the resection marker, and perform the resection.

From the foregoing it will be appreciated that, although specific embodiments have been described herein for purposes of illustration, various modifications may be made while remaining with the scope of the claimed technology.

Examples described herein may refer to various components as “coupled” or signals as being “provided to” or “received from” certain components. It is to be understood that in some examples the components are directly coupled one to another, while in other examples the components are coupled with intervening components disposed between them. Similarly, signal may be provided directly to and/or received directly from the recited components without intervening components, but also may be provided to and/or received from the certain components through intervening components.

What is claimed is:

1. A method comprising:
receiving position information for anatomical landmarks based on a position of a pointer having fiducials, the position information received at an augmented reality headset positioned to view the anatomical landmarks;
generating a surgical plan based on the position information; and
displaying, using the augmented reality headset, information to guide a device based on the surgical plan.

2. The method of claim 1, further comprising displaying, using the augmented reality headset, indication of the position information.

3. The method of claim 1, wherein the position information is further based on a fixed marker having fiducials, the augmented reality headset further positioned to view the fixed marker.

4. The method of claim 3, wherein the fixed marker is attached to anatomy.

5. A method comprising:
detecting, by an augmented reality device, a marker affixed to anatomy;
creating a coordinate system based in part on fiducials attached to the marker;
tracking a position of a pointer relative to the marker;
detecting an identification of a landmark, by the augmented reality device, based on the position of the pointer, the identification including landmark position information of the landmark;
determining a position of the landmark in the coordinate system;
generating an intra-operative plan based on the position of the landmark; and
providing surgical guidance using the augmented reality device based on the intra-operative plan.

6. The method of claim 5, further comprising:
selecting the landmark based on a particular surgical procedure.

7. The method of claim 5, further comprising:
prompting, by the augmented reality device, the identification of the landmark at least in part by instructing to have the pointer indicate the landmark.

8. The method of claim 5, wherein detecting the identification of the landmark is responsive to slowed or stopped motion of the pointer for a predetermined period.

9. The method of claim 5, further comprising:
prompting an additional identification of an additional landmark by the augmented reality device instructing to have the pointer indicate the landmark;
detecting the additional identification of the additional landmark based on the position of the pointer by the augmented reality device following a surgical intervention based on the surgical guidance, the additional identification including additional landmark position information of the additional landmark;
determining a position of the additional landmark in the coordinate system;
modifying the intra-operative plan based in part on the position of the additional landmark; and
providing the surgical guidance in the coordinate system based in part on the modified intra-operative plan.

10. The method of claim 9, further comprising:
generating an anatomical model of the anatomy based on the marker and the landmark;
updating the anatomical model based on the additional landmark; and
updating the surgical guidance based on the updated anatomical model.

11. The method of claim 10, wherein updating the surgical guidance is at least one of modifying a pre-operative plan or developing an additional portion of the pre-operative plan to update the intra-operative plan based on the updated anatomical model.

12. The method of claim 5, wherein providing the surgical guidance comprises:
tracking a current position of at least one of a cut plane, a guide, a surgical instrument, or a tool on a display; and
displaying the current position and a target position of the at least one of the cut plane, the guide, the surgical instrument, or the tool on the display.

13. The method of claim 12, wherein tracking the current position comprises:
detecting fiducials arranged in a predetermined pattern and attached to the at least one of the guide, the surgical instrument, or the tool by a depth camera of the augmented reality device;
identifying positions of the fiducials arranged in the predetermined pattern; and
determining position information of the at least one of the guide, the surgical instrument, or the tool based on the positions of the fiducials.

14. The method of claim 12, wherein providing the surgical guidance further comprises displaying at least one of a distance between the current position and the target position, or a direction to move the guide, the surgical instrument, or the tool.

15. The method of claim 5, further comprising:
obtaining marker position information of the marker and the landmark position information using data from a depth camera of the augmented reality device.

16. The method of claim 15, wherein the fiducials are first fiducials, the method further comprising:
detecting the first fiducials arranged in a predetermined pattern by the depth camera;
identifying positions of the first fiducials;
determining the marker position information of the marker based on the positions of the first fiducials;
detecting second fiducials arranged in a predetermined pattern and attached to the pointer by the depth camera;
identifying positions of the second fiducials; and
determining a location of the pointer relative to each marker based on the positions of the second fiducials.

17. The method of claim 5, further comprising:
calculating at least one axis or plane based on the determined position of the landmark; and
providing the surgical guidance on the augmented reality device based on the at least one axis or plane.

18. The method of claim 17, further comprising displaying the at least one axis or plane superimposed on the anatomy in augmented reality by the augmented reality device.

19. A system comprising:
a marker configured to be affixed to anatomy;
a pointer configured to point a landmark; and
an augmented reality device configured to:
detect the marker;
create a coordinate system based in part on fiducials attached to the marker;
prompt an identification of the landmark by instructing to have the pointer indicate the landmark;
detect the identification of the landmark responsive to an indication of the landmark;
determine a position of the landmark in the coordinate system;
generate an intra-operative plan based on the position of the landmark; and
provide surgical guidance using the augmented reality device based on the intra-operative plan.

20. The system of claim 19, wherein the pointer comprises a stylus configured to point the landmark.

21. The system of claim 19, wherein the augmented reality device is further configured to:
prompt an additional identification of an additional landmark by instructing to have the pointer indicate the landmark;
detect the additional identification of the additional landmark based on a position of the pointer following a surgical intervention based on the surgical guidance, the additional identification including additional landmark position information of the additional landmark;
determine a position of the additional landmark in the coordinate system;
modify the intra-operative plan based in part on the position of the additional landmark; and
provide the surgical guidance in the coordinate system based in part on the modified intra-operative plan.

22. The system of claim 19, further comprising fiducials attached to the pointer,
wherein the augmented reality device is configured to determine the position of the landmark based in part on relative positions of the fiducials attached to the pointer and the position of the marker.

23. The system of claim 19, wherein the augmented reality device comprises a depth camera configured to receive a depth image that includes a representation of fiducials relative to the device, and
wherein the augmented reality device is configured to determine the position of the landmark based on data received from the depth camera.

24. The system of claim 19, wherein the augmented reality device is further configured to:
calculate at least one axis or plane based on the determined position of the landmark; and
provide the surgical guidance on the augmented reality device based on the at least one axis or plane.

25. The system of claim 24, wherein the augmented reality device is configured to display the at least one axis or plane by superimposing the at least one axis or plane on the anatomy.

26. An augmented reality device comprising:
a processor; and
a non-transitory computer readable medium comprising instructions that, when executed, cause the processor to perform operations comprising:
detecting a marker affixed to anatomy;
creating a coordinate system based in part on fiducials attached to the marker;
prompting an identification of a landmark by an augmented reality device instructing to have a pointer indicate the landmark;
detecting the identification of the landmark based on a position of the pointer, the identification including landmark position information of the landmark;
determining a position of the landmark in the coordinate system;
generating an intra-operative plan based on the position of the landmark; and
providing surgical guidance based on the intra-operative plan.

27. The augmented reality device of claim 26, wherein the operations further comprise:
prompting an additional identification of an additional landmark by the augmented reality device instructing to have the pointer indicate the landmark;
detecting the additional identification of the additional landmark based on the position of the pointer following a surgical intervention based on the surgical guidance, the additional identification including additional landmark position information of the additional landmark;
determining a position of the additional landmark in the coordinate system;
modifying the intra-operative plan based in part on the position of the additional landmark; and
providing the surgical guidance in the coordinate system based in part on the modified intra-operative plan.

28. The augmented reality device of claim 26, wherein the operations further comprise selecting the landmark based on a surgical procedure.

29. The augmented reality device of claim 26, further comprising a depth camera, wherein the operations further comprise obtaining marker position information of the marker and the landmark position information using data from the depth camera.

30. The augmented reality device of claim 29, wherein the fiducials are first fiducials, and
wherein the operations further comprise:
detecting the first fiducials arranged in a predetermined pattern by the depth camera;
identifying positions of the first fiducials;
determining the marker position information of the marker based on the positions of the first fiducials;
detecting second fiducials arranged in a predetermined pattern and attached to the pointer by the depth camera;
identifying positions of the second fiducials; and
determining a position of the pointer relative to each marker based on the positions of the second fiducials.

31. The augmented reality device of claim 26, wherein the operations further comprise:
calculating at least one axis or plane based on the determined position of the landmark; and
providing the surgical guidance on the augmented reality device based on the at least one axis or plane.

32. The augmented reality device of claim 31, wherein the operations further comprise displaying the at least one axis or plane superimposed on the anatomy in augmented reality by the augmented reality device.
